(19) United States
(12) Patent Application Publication
(10) Pub. No.: US 2014/0322708 A1
(43) Pub. Date: Oct. 30, 2014

(54) METHOD FOR MEASURING SOMATIC DNA MUTATIONAL PROFILES

(71) Applicants: Jan Vijg, New York, NY (US); Michael Gundry, New York, NY (US); Wenge Li, Whippany, NJ (US)

(72) Inventors: Jan Vijg, New York, NY (US); Michael Gundry, New York, NY (US); Wenge Li, Whippany, NJ (US)

(73) Assignee: Albert Einstein College of Medicine of Yeshiva University, Bronx, NY (US)

(21) Appl. No.: 14/123,251

(22) PCT Filed: Jun. 1, 2012
(86) PCT No.: PCT/US12/40463

§ 371 Date: Jun. 9, 2014

Related U.S. Application Data
(60) Provisional application No. 61/492,580, filed on Jun. 2, 2011.

(51) Int. Cl. C12Q 1/68

(52) U.S. Cl. CPC C12Q 1/6869; G06F 19/22
USPC 435/6.11; 702/20

(57) ABSTRACT

Methods are provided for determining if an agent causes somatic mutations in a genome, and kits, systems and computer-readable medium therefor.

### CROSS-REFERENCE TO RELATED APPLICATIONS

This application claims benefit of U.S. Provisional Application No. 61/492,580, filed Jun. 2, 2011, the contents of which are hereby incorporated by reference.

### STATEMENT OF GOVERNMENT SUPPORT

This invention was made with government support under grant numbers ROI AG17242, RO1 AG20438, RO1 AG034421, and R21 AG030567 awarded by the National Institutes of Health. The government has certain rights in the invention.

### BACKGROUND OF THE INVENTION

Throughout this application various publications are referred to by number in parenthesis. Full citations for these references may be found at the end of the specification. The disclosures of these publications are hereby incorporated by reference in their entirety into the subject application to more fully describe the art to which the subject invention pertains.

Random alteration in the genome or epigenome of somatic cells is a cause of cancer and, possibly, aging. Such mutations or epimutations are a consequence of errors during restoration of a functional DNA molecule during repair or replication of a damaged DNA template. Damage to DNA is very frequent and induced by a variety of environmental and endogenous factors, varying from background radiation to the reactive oxygen species that arise as by-products of normal metabolism. In spite of its significance for health and disease there is very little information on the load of mutations and epimutations in somatic tissues of organisms. Because of their infrequent occurrence, i.e., varying from 10⁻⁶ to 10⁻² per locus depending on the type of DNA sequence involved, quantitation and characterization of these random events has been difficult. Large mutations, such as aneuploidy and chromosomal translocations can be analyzed by FISH, albeit at low resolution, i.e., >1 Mb. For smaller mutations, reporter assays have been the method of choice. For epimutations, such as random changes in DNA cytosine methylation, reporter systems are not even available. Reporter-based assays are also not representative for the genome as a whole and can never provide direct information about the mutation load of a cellular genome in a somatic tissue. Hence, while informative, DNA mutation loads at single loci are merely surrogate markers and cannot provide accurate predictions of risk based on a genome-wide DNA mutational profile. There is no technique in the art determining random mutation profiles by DNA sequencing. The present invention addresses this need.

### SUMMARY OF THE INVENTION

A method for determining if an agent increases somatic mutations in a genome of a cell, tissue, or subject exposed to the agent comprising:

a) amplifying a first sample of genomic nucleic acid obtained from a cell, tissue, or subject prior to the cell, tissue, or subject, respectively, being exposed to the agent;
b) either (i) randomly fragmenting the nucleic acid sample or (ii) generating a range of fragments of the nucleic acids from the sample amplified in step a) using one or more restriction enzymes, and then sequencing the resultant fragments;
c) mapping the fragments sequenced in step b) to a reference nucleic acid sequence;
d) comparing the sequences of fragments mapped in step c) to a corresponding portion of the reference nucleic acid sequence so as to identify, and, optionally, quantify, mutation(s), indels and/or genome rearrangements in the genomic nucleic acid of the first sample;
e) amplifying a second sample of genomic nucleic acid obtained from the cell, tissue, or subject, respectively, after the cell, tissue, or subject, respectively, has been exposed to the agent;
f) either (i) randomly fragmenting the nucleic acid sample or (ii) generating a range of fragments of the nucleic acids from the sample amplified in step e) using one or more restriction enzymes, and then sequencing the resultant fragments;
g) mapping the fragments sequenced in step f) to the reference nucleic acid sequence;
h) comparing the sequences of fragments mapped in step g) to a corresponding portion of the reference nucleic acid sequence so as to identify, and, optionally, quantify, mutation(s), indels and/or genome rearrangements in the genomic nucleic acid of the first sample;
i) comparing the number of mutations, indels and/or genome rearrangements identified or quantified in step h) with the number of mutations, indels and/or genome rearrangements identified or quantified in step d) for one or more sequences, or portion thereof, mapped in both step c) and step g),
wherein an increase in the number of mutations, indels and/or genome rearrangements identified or quantified in step h) compared to step d) indicates that the agent increases somatic mutations, indels and/or genome rearrangements in the genome of the cell, tissue, or subject, respectively, exposed to the agent, and wherein no change or a decrease in the number of mutations, indels and/or genome rearrangements identified or quantified in step h) compared to step d) indicates that the agent does not increase somatic mutations, indels and/or genome rearrangements in the genome of the cell, tissue, or subject, respectively, exposed to the agent.

A method for obtaining a mutation profile of a nucleic acid comprising:

a) amplifying a sample of the nucleic acid;
b) fragmenting the amplified sample and then sequencing in whole, or in part, those nucleic acids obtained in step a) which are of a predetermined range of lengths;
c) mapping fragments sequenced in step b) to a reference nucleic acid sequence; and
d) comparing the sequence of each fragment mapped in step c) to a corresponding portion of the reference nucleic acid sequence so as to identify mutation(s) in the nucleic acid,
thereby obtaining the mutation profile of the nucleic acid.

A method is also provided for determining if an agent increases somatic mutations in a genome of a cell, tissue, or subject exposed to the agent comprising:

a) amplifying a first sample of genomic nucleic acid obtained from a cell, tissue, or subject;
b) sequencing nucleic acids resulting from step a) either directly after randomly fragmenting the nucleic acids or after generating a range of fragments of the nucleic acids using one or more restriction enzymes;
c) mapping paired-end fragments sequenced in step b) to a reference nucleic acid sequence;
d) comparing the sequences of paired-end fragments mapped in step c) to a corresponding portion of the reference nucleic acid sequence so as to identify mutation(s), indels and/or genome rearrangements in the genomic nucleic acid of the first sample;
e) amplifying a second sample of genomic nucleic acid obtained from the cell, tissue, or subject, respectively, after the cell, tissue, or subject, respectively, has been exposed to the agent;
f) sequencing nucleic acids resulting from step e) either directly after randomly fragmenting the nucleic acids or after generating a range of fragments of predetermined lengths of the nucleic acids using one or more restriction enzymes;
g) mapping paired-end fragments sequenced. in step f) to the reference nucleic acid sequence;
h) comparing the sequences of paired-end fragments mapped in step g) to a corresponding portion of the reference nucleic acid sequence so as to identify mutation(s), indels and/or genome rearrangements in the genomic nucleic acid of the second sample;
i) comparing the number of mutations, indels and/or genome rearrangements identified in step h) with the number of mutations, indels and/or genome rearrangements identified in step d) for one or more sequences, or portion thereof mapped in both step c) and step g),
wherein an increase in the number of mutations, indels and/or genome rearrangements identified in step h) compared to step d) indicates that the agent increases somatic mutations, indels and/or genome rearrangements in the genome of the cell, tissue, or subject, respectively, exposed to the agent, and wherein no change or a decrease in the number of mutations, indels and/or genome rearrangements identified in step h) compared to step d) indicates that the agent does not increase somatic mutations, indels and/or genome rearrangements in the genome of the cell, tissue, or subject, respectively, exposed to the agent.

A method is also provided for obtaining a mutation profile of a nucleic acid comprising:

a) amplifying a sample of the nucleic acid;
b) sequencing in whole, or in part, those nucleic acids obtained in step a) which are of a predetermined range of lengths;
c) mapping paired-end fragments sequenced in step b) to a reference nucleic acid sequence; and
d) comparing the sequence of each fragment mapped in step c) to a corresponding portion of the reference nucleic acid sequence so as to identify mutation(s) in the nucleic acid,
thereby obtaining the mutation profile of the nucleic acid.

Also provided is a method for determining if an agent increases somatic mutations in a genome of a cell, tissue or subject exposed to the agent, comprising:

a) contacting a first sample of genomic nucleic acid, obtained from the cell, tissue, or subject before exposure to the agent, with a restriction enzyme under conditions permitting the restriction enzyme to cleave the genomic nucleic acid into a first plurality of fragments;
b) sequencing in whole, or in part, fragments produced in step a) of a predetermined length range;
c) mapping paired-end fragments sequenced in step b) to a reference nucleic acid sequence;
d) comparing the sequences of the paired-end fragments mapped in step c) to a corresponding portion of the reference nucleic acid sequence so as to identify mutation(s), indels and/or genome rearrangements in the genomic nucleic acid;
e) contacting a second sample of genomic nucleic acid, obtained from the cell, tissue or subject after the cell, tissue or subject, respectively, has been exposed to the agent, with a restriction enzyme under conditions permitting the restriction enzyme to cleave the genomic nucleic acid into a second plurality of fragments;
f) sequencing in whole, or in part, fragments produced in step e) which are of the predetermined length range;
g) mapping paired-end fragments sequenced in step f) to the reference nucleic acid sequence;
h) comparing the sequences of fragments mapped in step g) to a corresponding portion of the reference nucleic acid sequence so as to identify mutation(s), indels and/or genome rearrangements in the genomic nucleic acid of the second sample after exposure to the agent; and
i) comparing the number of mutations, indels and/or genome rearrangements identified in step h) with the number identified in step d) for one or more sequences, or portion thereof, mapped in both step c) and step g),
wherein an increase in the number of mutations, indels and/or genome rearrangements identified in step h) compared to step d) indicates that the agent increases somatic mutations, indels and/or genome rearrangements in the genome of the cell, tissue or subject, respectively, exposed to the agent, and wherein no change or a decrease in the number of mutations, indels and/or genome rearrangements identified in step h) compared to step d) indicates that the agent does not increase somatic mutations, indels and/or genome rearrangements in the genome of the cell, tissue, or subject, respectively, exposed to the agent.

Also provided is a method for obtaining a mutation profile of a nucleic acid comprising:

a) contacting the nucleic acid with a restriction enzyme under conditions permitting the restriction enzyme to cleave the nucleic acid into a plurality of paired-end fragments;
b) sequencing in whole, or in part, fragments obtained in step a) which are of a predetermined range of lengths;
c) mapping paired-end fragments sequenced in step b) to a reference nucleic acid sequence; and
d) comparing the sequence of each fragment mapped in step c) to a corresponding portion of the reference nucleic acid sequence so as to identify mutation(s) in the nucleic acid,
thereby obtaining the mutation profile of the nucleic acid.

Also provided is a method for determining if an agent increases somatic mutations in a genome of a cell, tissue or subject exposed to the agent, comprising:

a) accessing, using one or more processors, a first set of data from a database, the first set of data being a reference nucleic acid sequence;
b) mapping to the reference nucleic acid sequence, using one or more processors, sequences of paired-end fragments obtained and sequenced from the genome of the cell, tissue or subject or obtained and sequenced from a nucleic acid amplified from the genome of the cell, tissue or subject, before the genome has been exposed to the agent;
c) comparing, using one or more processors, the sequences of the mapped paired-end fragment sequences to corresponding portions of the reference nucleic acid sequence thereby generating a second set of data which comprises all mutations, indels and/or genome rearrangements identified in the genome of the cell, tissue or subject relative to the reference nucleic acid sequence;
d) accessing, using one or more processors, the first set of data from the database, the first set of data being the reference nucleic acid sequence;
e) after the genome has been exposed to the agent, mapping to the reference nucleic acid sequence, using one or more processors, sequences of paired-end fragments obtained and sequenced from the genome of the cell, tissue or subject or obtained and sequenced from a nucleic acid amplified from the genome of the cell, tissue or subject;
f) comparing, using one or more processors, the sequences of mapped paired-end fragment sequences to corresponding portions of the reference nucleic acid sequence thereby generating a third set of data which comprises mutations, indels and/or genome rearrangements identified in the genome of the cell, tissue or subject, after the genome has been exposed to the agent, relative to the reference nucleic acid sequence; and
g) comparing, using one or more processors, the second set of data and the third set of data, wherein an increase in the number of mutations, indels and/or genome rearrangements identified in the third set of data compared to the second set of data indicates that the agent increases somatic mutations, indels and/or genome rearrangements in the genome of the cell, tissue or subject, respectively, exposed to the agent, and wherein no change or a decrease in the number of mutations, indels and/or genome rearrangements in the third set of data compared to the second set of data indicates that the agent does not increase somatic mutations, indels and/or genome rearrangements in the genome of the cell, tissue, or subject, respectively, exposed to the agent.

Also provided is a system for determining if an agent increases somatic mutations in a genome of a cell, tissue or subject exposed to the agent, comprising: one or more data processing apparatus; and

a computer-readable medium coupled to the one or more data processing apparatus having instructions stored thereon which, when executed by the one or more data processing apparatus, cause the one or more data processing apparatus to perform a method comprising:
a) accessing, using one or more processors, a first set of data from a database, the first set of data being a reference nucleic acid sequence;
b) mapping to the reference nucleic acid sequence, using one or more processors, sequences of paired-end fragments obtained and sequenced from the genome of the cell, tissue or subject or obtained and sequenced from a nucleic acid amplified from the genome of the cell, tissue or subject, before the genome has been exposed to the agent;
c) comparing, using one or more processors, the sequences of the mapped paired-end fragment sequences to corresponding portions of the reference nucleic acid sequence thereby generating a second set of data which comprises all mutations, indels and/or genome rearrangements identified in the genome of the cell, tissue or subject relative to the reference nucleic acid sequence;
d) accessing, using one or more processors, the first set of data from the database, the first set of data being the reference nucleic acid sequence;
e) after the genome has been exposed to the agent, mapping to the reference nucleic acid sequence, using one or more processors, sequences of paired-end fragments obtained and sequenced from the genome of the cell, tissue or subject or obtained and sequenced from a nucleic acid amplified from the genome of the cell, tissue or subject;
f) comparing, using one or more processors, the sequences of mapped paired-end fragment sequences to corresponding portions of the reference nucleic acid sequence thereby generating a third set of data which comprises mutations, indels and/or genome rearrangements identified in the genome of the cell, tissue or subject, after the genome has been exposed to the agent, relative to the reference nucleic acid sequence; and
g) comparing, using one or more processors, the second set of data and the third set of data, wherein an increase in the number of mutations, indels and/or genome rearrangements identified in the third set of data compared to the second set of data indicates that the agent increases somatic mutations, indels and/or genome rearrangements in the genome of the cell, tissue or subject, respectively, exposed to the agent, and wherein no change or a decrease in the number of mutations, indels and/or genome rearrangements in the third set of data compared to the second set of data indicates that the agent does not increase somatic mutations, indels and/or genome rearrangements in the genome of the cell, tissue, or subject, respectively, exposed to the agent.

Also provided is a computer-readable medium comprising instructions stored thereon which, when executed by a data processing apparatus, causes the data processing apparatus to perform a method comprising:

a) accessing, using one or more processors, a first set of data from a database, the first set of data being a reference nucleic acid sequence;
b) mapping to the reference nucleic acid sequence, using one or more processors, sequences of paired-end fragments obtained and sequenced from the genome of the cell, tissue or subject or obtained and sequenced from a nucleic acid amplified from the genome of the cell, tissue or subject, before the genome has been exposed to the agent;
c) comparing, using one or more processors, the sequences of the mapped paired-end fragment sequences to corresponding portions of the reference nucleic acid sequence thereby generating a second set of data which comprises all mutations, indels and/or genome rearrangements identified in the genome of the cell, tissue or subject relative to the reference nucleic acid sequence;
d) accessing, using one or more processors, the first set of data from the database, the first set of data being the reference nucleic acid sequence;
e) after the genome has been exposed to the agent, mapping to the reference nucleic acid sequence, using one or more processors, sequences of paired-end fragments obtained and sequenced from the genome of the cell, tissue or subject or obtained and sequenced from a nucleic acid amplified from the genome of the cell, tissue or subject;
f) comparing, using one or more processors, the sequences of mapped paired-end fragment sequences to corresponding portions of the reference nucleic acid sequence thereby generating a third set of data which comprises mutations, indels and/or genome rearrangements identified in the genome of the cell, tissue or subject, after the genome has been exposed. to the agent, relative to the reference nucleic acid sequence; and
g) comparing, using one or more processors, the second set of data and the third set of data, wherein an increase in the number of mutations, indels and/or genome rearrangements identified in the third set of data compared to the second set of data indicates that the agent increases somatic mutations, indels and/or genome rearrangements in the genome of the cell, tissue or subject, respectively, exposed to the agent, and wherein no change or a decrease in the number of mutations, indels and/or genome rearrangements in the third set of data compared to the second set of data indicates that the agent does not increase somatic mutations, indels and/or genome rearrangements in the genome of the cell, tissue, or subject, respectively, exposed to the agent.

A kit is provided comprising reagents and protocol instructions for performing one of the instant methods.

Also provided is a method for determining if a subject is susceptible to a mutagenic agent that increases somatic mutations in a genome of a cell, tissue, or sample exposed to the agent comprising:

a) amplifying a first sample of genomic nucleic acid obtained from a cell, tissue, or sample subject prior to the cell, tissue, or sample, respectively, being exposed to the agent;
b) either (i) randomly fragmenting the nucleic acid sample or (ii) generating a range of fragments of the nucleic acids from the sample amplified in step a) using one or more restriction enzymes, and then sequencing the resultant fragments;
c) napping the fragments sequenced in step b) to a reference nucleic acid sequence;
d) comparing the sequences of fragments mapped in step c) to a corresponding portion of the reference nucleic acid sequence so as to identify, and, optionally, quantify, mutation(s), indels and/or genome rearrangements in the genomic nucleic acid of the first sample;
e) amplifying a second sample of genomic nucleic acid obtained from the cell, tissue, or sample, respectively, after the cell, tissue, or sample, respectively, has been exposed to the agent;
f) either (i) randomly fragmenting the nucleic acid sample or (ii) generating a range of fragments of the nucleic acids from the sample amplified in step e) using one or more restriction enzymes, and then sequencing the resultant fragments;
g) mapping the fragments sequenced in step f) to the reference nucleic acid sequence;
h) comparing the sequences of fragments mapped in step g) to a corresponding portion of the reference nucleic acid sequence so as to identify, and, optionally, quantify, mutation(s), indels and/or genome rearrangements in the genomic nucleic acid of the first sample;
i) comparing the number of mutations, indels and/or genome rearrangements identified or quantified in step h) with the number of mutations, indels and/or genome rearrangements identified or quantified in step d) for one or more sequences, or portion thereof, mapped in both step c) and step g),
wherein an increase in the number of mutations, indels and/or genome rearrangements identified or quantified in step h) compared to step d) in excess of a predetermined control level indicates that the subject is susceptible to the mutagenic agent and wherein the number of mutations, indels and/or genome rearrangements identified or quantified in step h) compared to step d) at or below a predetermined control level indicates that the subject is not susceptible to the mutagenic agent.

An apparatus system for determining if an agent increases somatic mutations in a genome of a cell, tissue or subject exposed to the agent, comprising:

one or nucleic acid more sequencing machine(s) and, optionally, one or more data processing apparatus and a computer-readable medium coupled to the one or more data processing apparatus having instructions stored thereon which, when executed by the one or more data processing apparatus, cause the one or more data processing apparatus to perform a method comprising:
a) accessing, using one or more processors, a first set of data from a database, the first set of data being a reference nucleic acid sequence;
b) mapping to the reference nucleic acid sequence, using one or more processors, sequences of fragments obtained and sequenced from the genome of the cell, tissue or subject or obtained and sequenced from a nucleic acid amplified from the genome of the cell, tissue or subject, before the genome has been exposed to the agent, wherein the fragments are sequenced by the one or more sequencing machine(s);
c) comparing, using one or more processors, the sequences of the mapped fragment sequences to corresponding portions of the reference nucleic acid sequence thereby generating a second set of data which comprises all mutations, indels and/or genome rearrangements identified in the genome of the cell, tissue or subject relative to the reference nucleic acid sequence;
d) accessing, using one or more processors, the first set of data from the database, the first set of data being the reference nucleic acid sequence;
e) after the genome has been exposed to the agent, mapping to the reference nucleic acid sequence, using one or more processors, sequences of fragments obtained and sequenced from the genome of the cell, tissue or subject or obtained and sequenced from a nucleic acid amplified from the genome of the cell, tissue or subject, wherein the fragments are sequenced by the one or more sequencing machine(s);
f) comparing, using one or more processors, the sequences of mapped fragment sequences to corresponding portions of the reference nucleic acid sequence thereby generating a third set of data which comprises mutations, indels and/or genome rearrangements identified in the genome of the cell, tissue or subject, after the genome has been exposed to the agent, relative to the reference nucleic acid sequence; and
g) comparing, using one or more processors, the second set of data and the third set of data, wherein an increase in the number of mutations, indels and/or genome rearrangements identified in the third set of data compared to the second set of data indicates that the agent increases somatic mutations, indels and/or genome rearrangements in the genome of the cell, tissue or subject, respectively, exposed to the agent, and wherein no change or a decrease in the number of mutations, indels and/or genome rearrangements in the third set of data compared to the second set of data indicates that the agent does not increase somatic mutations, indels and/or genome rearrangements in the genome of the cell, tissue, or subject, respectively, exposed to the agent.

### BRIEF DESCRIPTION OF THE DRAWINGS

FIG. 1A-1B: Somatic mutation detection using single cell sequencing. (1A). Somatic mutations in tissues are rare and therefore found only in single sequencing reads from which they are routinely filtered out as sequencing errors during post-alignment processing. Adopting a single cell approach overcomes this limitation by transforming each somatic event into a consensus variant call. (1B). Schematic depiction of one embodiment of the single cell sequencing protocol.

FIG. 2A-2B: Mutant read frequencies on chr2L and chrX. (2A). Histogram of the mutant read frequencies for 498 somatic point mutations on chr2L. The superimposed line demonstrates a normal distribution with mean of 25 and the observed standard deviation of 21. (2B). Histogram of the mutant read frequencies for 227 somatic point mutations on chrX. The superimposed line demonstrates a normal distribution with mean of 50 and the observed standard deviation of 22.

FIG. 3A-3B: (Genome-wide sequence coverage and mutation localization. (3A) Single S2 control cell #1. (3B) Single S2 ENU-treated cell #3.

FIG. 4A-4C: Somatic point mutation frequencies and spectra. (4A). Somatic mutation frequencies for the nine single cells. (4B). Mutation spectra for the control and ENU-treated S2 and MEF cells. (4C). Strand of origin for ENU-induced mutations within genes.

FIG. 5: Locus dropout. Whole genome amplification (WGA) introduces a considerable amount of coverage bias due to the unequal amplification of different loci. In order to proceed with single cells that had the greatest fraction of loci represented with sufficient coverage, a SYBR-Green real-time PCR assay targeting eight loci was used. 2 ng of WGA DNA from each single cell was input into each reaction and the resultant Ct value was compared to that obtained with 2 ng of input DNA from an unamplified control sample. Using the differences in Ct values, the relative abundance of each locus was estimated. The chart in FIG. 5 shows data from a screening performed on 11 WGA MEFs. Samples with (**) denote those that were chosen for sequencing.

FIG. 6A-6B: Somatic point mutation validation. (6A). Integrated Genornomics Viewer (IGV) window showing a somatic mutation identified in an ENU-treated cell (top panel) but not found in the population (bottom panel). (6B). The same mutation was validated using Sanger sequencing.

FIG. 7: S2 cell karyotype. Metaphase FISH was performed on the S2 cell line. Out of 52 cells analyzed, none displayed a 2n karyotype. Observed was also the G:C->A:T transition, which did not localize at CpG sites and hence does not appear to be a product of spontaneous deamination as genomic DNA methylation levels in the fly are below 0.5%. The spontaneous mutation spectra observed in our single S2 cells is different than that observed in accumulation line experiments, perhaps due to different repair mechanisms operating in the germ-line vs. the S2 cell line.

### DETAILED DESCRIPTION OF THE INVENTION

A method is provided for determining if an agent increases somatic mutations in a genome of a cell, tissue, or subject exposed to the agent comprising:

a) amplifying a first sample of genomic nucleic acid obtained from a cell, tissue, or subject prior to the cell, tissue, or subject, respectively, being exposed to the agent;
b) either (i) randomly fragmenting the nucleic acid sample into fragments or (ii) generating a range of fragments of the nucleic acids from the sample amplified in step a) using one or more restriction enzymes, and then sequencing the resultant fragments;
c) mapping the fragments sequenced in step b) to a reference nucleic acid sequence;
d) comparing the sequences of fragments mapped in step c) to a corresponding portion of the reference nucleic acid sequence so as to identify, and, optionally, quantify, mutation(s), indels and/or genome rearrangements in the genomic nucleic acid. of the first sample;
e) amplifying a second sample of genomic nucleic acid obtained from the cell, tissue, or subject, respectively, after the cell, tissue, or subject, respectively, has been exposed to the agent;
i) either (i) randomly fragmenting the nucleic acid sample into fragments or (ii) generating a range of fragments of the nucleic acids from the sample amplified in step e) using one or more restriction enzymes, and then sequencing the resultant fragments;
g) mapping the fragments sequenced in step f) to the reference nucleic acid sequence;
h) comparing the sequences of fragments mapped in step g) to a corresponding portion of the reference nucleic acid sequence so as to identify, and, optionally, quantify, mutation(s), indels and/or genome rearrangements in the genomic nucleic acid of the first sample;
i) comparing the number of mutations, indels and/or genome rearrangements identified or quantified in step h) with the number of mutations, indels and/or genome rearrangements identified or quantified in step d) for one or more sequences, or portion thereof, mapped in both step c) and step g),
wherein an increase in the number of mutations, indels and/or genome rearrangements identified or quantified in step h) compared to step d) indicates that the agent increases somatic mutations, indels and/or genome rearrangements in the genome of the cell, tissue, or subject, respectively, exposed to the agent, and wherein no change or a decrease in the number of mutations, indels and/or genome rearrangements identified or quantified in step h) compared to step d) indicates that the agent does not increase somatic mutations, indels and/or genome rearrangements in the genome of the cell, tissue, or subject, respectively, exposed to the agent.

In an embodiment of the method, in step b) and/or in step f) the fragments are sequenced by paired-end sequencing. In an embodiment of the method further comprises in each of steps b) and f) size-selecting fragments before sequencing. In an embodiment of the method, in step b) sequencing a locus on the fragments a plurality of times and selecting a consensus sequence of the resultant plurality of sequencing results as the fragment sequence mapped in step c) and compared to in step d). In an embodiment of the method, in step f) sequencing a locus on the fragments a plurality of times and selecting a consensus sequence of the resultant plurality of sequencing results as the fragment sequence mapped in step g) and compared to in step h).

A method is also provided for obtaining a mutation profile of a nucleic acid comprising:

a) amplifying a sample of the nucleic acid;
b) fragmenting the amplified sample and then sequencing in whole, or in part, those nucleic acids obtained in step a) which are of a predetermined range of lengths;
c) mapping fragments sequenced in step b) to a reference nucleic acid sequence; and
d) comparing the sequence of each fragment mapped in step c) to a corresponding portion of the reference nucleic acid sequence so as to identify mutation(s) in the nucleic acid,
thereby obtaining the mutation profile of the nucleic acid.

In an embodiment of the method, in step b) the fragments are sequenced by paired-end sequencing. In an embodiment, the method further comprises in step b) size-selecting fragments before sequencing.

In an embodiment of the methods herein disclosed, the amplifying is whole genome amplification. In an embodiment of the methods herein disclosed, the methods further comprise screening the amplified genome for locus dropout. In an embodiment of the methods herein disclosed, screening the amplified genome for locus dropout is effected by using primer pairs distributed over different chromosomes and qPCR. In an embodiment of the methods herein disclosed, the subject is a human subject.

In an embodiment of the methods herein disclosed, the subject has cancer and the agent is a chemotherapeutic.

A method is also provided for determining if an agent increases somatic mutations in a genome of a cell, tissue or subject exposed to the agent, comprising:

a) contacting a first sample of genomic nucleic acid, obtained from the cell, tissue, or subject before exposure to the agent, with a restriction enzyme under conditions permitting the restriction enzyme to cleave the genomic nucleic acid into a first plurality of fragments;
b) sequencing in whole, or in part, fragments produced in step a) of a predetermined length range;
c) mapping paired-end fragments sequenced in step b) to a reference nucleic acid sequence;
d) comparing the sequences of the paired-end fragments mapped in step c) to a corresponding portion of the reference nucleic acid sequence so as to identify mutation(s), indels and/or genome rearrangements in the genomic nucleic acid;
e) contacting a second sample of genomic nucleic acid, obtained from the cell, tissue or subject after the cell, tissue or subject, respectively, has been exposed to the agent, with a restriction enzyme under conditions permitting the restriction enzyme to cleave the genomic nucleic acid into a second plurality of fragments;
f) sequencing in whole, or in part, fragments produced in step e) which are of the predetermined length range;
g) mapping paired-end fragments sequenced in step f) to the reference nucleic acid sequence;
h) comparing the sequences of fragments mapped in step g) to a corresponding portion of the reference nucleic acid sequence so as to identify mutation(s), indels and/or genome rearrangements in the genomic nucleic acid of the second sample after exposure to the agent; and
i) comparing the number of mutations, indels and/or genome rearrangements identified in step h) with the number identified in step d) for one or more sequences, or portion thereof, mapped in both step c) and step g),
wherein an increase in the number of mutations, indels and/or genome rearrangements identified in step h) compared to step d) indicates that the agent increases somatic mutations, indels and/or genome rearrangements in the genome of the cell, tissue or subject, respectively, exposed to the agent, and wherein no change or a decrease in the number of mutations, indels and/or genome rearrangements identified in step h) compared to step d) indicates that the agent does not increase somatic mutations, indels and/or genome rearrangements in the genome of the cell, tissue, or subject, respectively, exposed to the agent.

A method is also provided for obtaining a mutation profile of a nucleic acid comprising:

a) contacting the nucleic acid with a restriction enzyme under conditions permitting the restriction enzyme to cleave the nucleic acid into a plurality of paired-end fragments;
b) sequencing in whole, or in part, fragments obtained in step a) which are of a predetermined range of lengths;
c) mapping paired-end fragments sequenced in step b) to a reference nucleic acid sequence; and
d) comparing the sequence of each fragment mapped in step c) to a corresponding portion of the reference nucleic acid sequence so as to identify mutation(s) in the nucleic acid,
thereby obtaining the mutation profile of the nucleic acid.

In an embodiment, the method further comprises analyzing the mutation profile obtained by dividing the number of mutations identified in step d) by the total number of base pairs of the fragments sequenced in step b) so as to obtain a mutation frequency value or a base-pair mutation rate.

In an embodiment, the method further comprises comparing the mutation frequency value with a predetermined mutation frequency value obtained from a control so as to identify whether the mutation profile of the nucleic acid comprises more mutations than the control.

In an embodiment, the method further comprises comparing the number of mutation(s), indels and/or genome rearrangements identified with a predetermined number of mutations, indels and/or genome rearrangements obtained from a control, so as to identify whether the mutation profile of the nucleic acid comprises more mutations, indels and/or genome rearrangements than the control.

In an embodiment of the methods disclosed herein involving exposure to an agent, the time between the end of exposure of the cell, tissue or subject to the agent and the beginning of step e) is at least one hour, at least one day, at least one week, at least one month or at least one year.

In an embodiment of the methods disclosed herein, the genomic nucleic acid is amplified prior to step a).

In an embodiment of the methods disclosed herein, the nucleic acid is amplified with a polymerase chain reaction (PCR).

In an embodiment of the methods disclosed herein, the nucleic acid is amplified by whole genome amplification using multiple displacement amplification.

In an embodiment of the methods disclosed herein, in steps d) and h), the number of mutations is quantified.

In an embodiment of the methods disclosed herein, the methods further comprise discounting all rearrangement artifacts from the number of mutations quantified.

In an embodiment of the methods disclosed herein, the nucleic acid is a genomic nucleic acid.

In an embodiment of the methods disclosed herein, the nucleic acid is obtained from a somatic cell.

In an embodiment of the methods disclosed herein, the nucleic acid is a single cell genome.

In an embodiment of the methods disclosed herein, the restriction enzyme is HindIII, PstI or MseI.

In an embodiment of the methods disclosed herein, the nucleic acid is obtained from a human subject.

In an embodiment of the methods disclosed herein, the reference nucleic acid sequence is a human genome set forth in hg19 or is a custom reference sequence determined from a predetermined cell, tissue or subject of the same type as the cell, tissue or subject the nucleic acid sample was obtained from.

In an embodiment of the methods disclosed herein, the methods further comprise, after mapping paired-end sequenced fragments, discarding sequences having a mapping quality score below a predetermined value prior to comparing the sequences of the remaining fragments to the corresponding portions of the reference nucleic acid sequence.

In an embodiment of the methods disclosed herein, the methods further comprise, after mapping paired-end sequenced fragments, discarding chimeric sequences, wherein a sequence is determined as chimeric through application of an algorithm that uses an in silico digestion to define a chimeric signature as occurring between two fragments selected for during restriction digestion and subsequent predetermined length selection.

In an embodiment of the methods disclosed herein, the methods further comprise comparing sequences displaying evidence of a genome rearrangement that were not defined as chimeric to the total number of sequencing reads to calculate the rearrangement mutation frequency.

In an embodiment of the methods disclosed herein, the mutations are small indels or point mutations that remain after applying an artifact filtering algorithm.

In an embodiment of the methods disclosed herein, the subject is a human subject and has cancer.

In an embodiment of the methods disclosed herein, the agent is a chemotherapeutic. In an embodiment of the methods disclosed herein, the agent is a chemical having a mass of 1000 daltons or less. In an embodiment of the methods disclosed herein, the chemical comprises an organic chemical.

In an embodiment of the methods disclosed herein, the agent comprises a radioactive agent. In an embodiment of the methods disclosed herein, the agent comprises a virus. In an embodiment of the methods disclosed herein, the agent comprises a transposon.

In an embodiment of the methods disclosed herein, the sample comprises a blood sample. In an embodiment of the methods disclosed herein, the sample comprises a tissue sample. In an embodiment of the methods disclosed herein, the sample comprises a cancer cell. In an embodiment of the methods disclosed herein, the sample comprises a stem cell.

A method is also provided for determining if an agent increases somatic mutations in a genome of a cell, tissue or subject exposed to the agent, comprising:

a) accessing, using one or more processors, a first set of data from a database, the first set of data being a reference nucleic acid sequence;
b) mapping to the reference nucleic acid sequence, using one or more processors, sequences of fragments obtained and sequenced from the genome of the cell, tissue or subject or obtained and sequenced from a nucleic acid amplified from the genome of the cell, tissue or subject, before the genome has been exposed to the agent;
c) comparing, using one or more processors, the sequences of the mapped fragment sequences to corresponding portions of the reference nucleic acid sequence thereby generating a second set of data which comprises all mutations, indels and/or genome rearrangements identified in the genome of the cell, tissue or subject relative to the reference nucleic acid sequence;
d) accessing, using one or more processors, the first set of data from the database, the first set of data being the reference nucleic acid sequence;
e) after the genome has been exposed to the agent, mapping to the reference nucleic acid sequence, using one or more processors, sequences of fragments obtained and sequenced from the genome of the cell, tissue or subject or obtained and sequenced from a nucleic acid amplified from the genome of the cell, tissue or subject;
f) comparing, using one or more processors, the sequences of mapped fragment sequences to corresponding portions of the reference nucleic acid sequence thereby generating a third set of data which comprises mutations, indels and/or genome rearrangements identified in the genome of the cell, tissue or subject, after the genome has been exposed to the agent, relative to the reference nucleic acid sequence; and
g) comparing, using one or more processors, the second set of data and the third set of data, wherein an increase in the number of mutations, indels and/or genome rearrangements identified in the third set of data compared to the second set of data indicates that the agent increases somatic mutations, indels and/or genome rearrangements in the genome of the cell, tissue or subject, respectively, exposed to the agent, and wherein no change or a decrease in the number of mutations, indels and/or genome rearrangements in the third set of data compared to the second set of data indicates that the agent does not increase somatic mutations, indels and/or genome rearrangements in the genome of the cell, tissue, or subject, respectively, exposed to the agent.

A system is provided for determining if an agent increases somatic mutations in a genome of a cell, tissue or subject exposed to the agent, comprising:

one or more data processing apparatus; and
a computer-readable medium coupled to the one or more data processing apparatus having instructions stored thereon which, when executed by the one or more data processing apparatus, cause the one or more data processing apparatus to perform a method comprising:
a) accessing, using one or more processors, a first set of data from a database, the first set of data being a reference nucleic acid sequence;
b) mapping to the reference nucleic acid sequence, using one or more processors, sequences of fragments obtained and sequenced from the genome of the cell, tissue or subject or obtained and sequenced from a nucleic acid amplified from the genome of the cell, tissue or subject, before the genome has been exposed to the agent;
c) comparing, using one or more processors, the sequences of the mapped fragment sequences to corresponding portions of the reference nucleic acid sequence thereby generating a second set of data which comprises all mutations, indels and/or genome rearrangements identified in the genome of the cell, tissue or subject relative to the reference nucleic acid sequence;
d) accessing, using one or more processors, the first set of data from the database, the first set of data being the reference nucleic acid sequence;
e) after the genome has been exposed to the agent, mapping to the reference nucleic acid sequence, using one or more processors, sequences of fragments obtained and sequenced from the genome of the cell, tissue or subject or obtained and sequenced from a nucleic acid amplified from the genome of the cell, tissue or subject;
f) comparing, using one or more processors, the sequences of mapped fragment sequences to corresponding portions of the reference nucleic acid sequence thereby generating a third set of data which comprises mutations, indels and/or genome rearrangements identified in the genome of the cell, tissue or subject, after the genome has been exposed to the agent, relative to the reference nucleic acid sequence; and
g) comparing, using one or more processors, the second set of data and the third set of data, wherein an increase in the number of mutations, indels and/or genome rearrangements identified in the third set of data compared to the second set of data indicates that the agent increases somatic mutations, indels and/or genome rearrangements in the genome of the cell, tissue or subject, respectively, exposed to the agent, and wherein no change or a decrease in the number of mutations, indels and/or genome rearrangements in the third set of data compared to the second set of data indicates that the agent does not increase somatic mutations, indels and/or genome rearrangements in the genome of the cell, tissue, or subject, respectively, exposed to the agent.

A computer-readable medium is provided comprising instructions stored thereon which, when executed by a data processing apparatus, causes the data processing apparatus to perform a method comprising:

a) accessing, using one or more processors, a first set of data from a database, the first set of data being a reference nucleic acid sequence;
b) mapping to the reference nucleic acid sequence, using one or more processors, sequences of fragments obtained and sequenced from the genome of the cell, tissue or subject or obtained and sequenced from a nucleic acid amplified from the genome of the cell, tissue or subject, before the genome has been exposed to the agent;
c) comparing, using one or more processors, the sequences of the mapped fragment sequences to corresponding portions of the reference nucleic acid sequence thereby generating a second set of data which comprises all mutations, indels and/or genome rearrangements identified in the genome of the cell, tissue or subject relative to the reference nucleic acid sequence;
d) accessing, using one or more processors, the first set of data from the database, the first set of data being the reference nucleic acid sequence;
e) after the genome has been exposed to the agent, mapping to the reference nucleic acid sequence, using one or more processors, sequences of fragments obtained and sequenced from the genome of the cell, tissue or subject or obtained and sequenced from a nucleic acid amplified from the genome of the cell, tissue or subject;
f) comparing, using one or more processors, the sequences of mapped fragment sequences to corresponding portions of the reference nucleic acid sequence thereby generating a third set of data which comprises mutations, indels and/or genome rearrangements identified in the genome of the cell, tissue or subject, after the genome has been exposed to the agent, relative to the reference nucleic acid sequence; and
g) comparing, using one or more processors, the second set of data and the third set of data, wherein an increase in the number of mutations, indels and/or genome rearrangements identified in the third set of data compared to the second set of data indicates that the agent increases somatic mutations, indels and/or genome rearrangements in the genome of the cell, tissue or subject, respectively, exposed to the agent, and wherein no change or a decrease in the number of mutations, indels and/or genome rearrangements in the third set of data compared to the second set of data indicates that the agent does not increase somatic mutations, indels and/or genome rearrangements in the genome of the cell, tissue, or subject, respectively, exposed to the agent.

In an embodiment of the method, the system or the computer-readable medium, the fragments sequenced in steps b) and/or e) are sequenced by paired-end sequencing.

In an embodiment of the methods disclosed herein, the fragments represent up to 2% of the genome. In an embodiment of the methods disclosed herein, the fragments represent up to 1% of the genome.

In an embodiment of the methods disclosed herein, the method further comprises obtaining the sample of the nucleic acid from the subject prior to step a).

In an embodiment the method for determining if a subject is susceptible to a mutagenic agent that increases somatic mutations in a genome of a cell, tissue, or sample exposed to the agent comprising:

a) amplifying a first sample of genomic nucleic acid obtained from a cell, tissue, or sample subject prior to the cell, tissue, or sample, respectively, being exposed to the agent;
b) either (i) randomly fragmenting the nucleic acid sample or (ii) generating a range of fragments of the nucleic acids from the sample amplified in step a) using one or more restriction enzymes, and then sequencing the resultant fragments;
c) mapping the fragments sequenced in step b) to a reference nucleic acid sequence;
d) comparing the sequences of fragments mapped in step c) to a corresponding portion of the reference nucleic acid sequence so as to identify, and, optionally, quantify, mutation(s), indels and/or genome rearrangements in the genomic nucleic acid of the first sample;
e) amplifying a second sample of genomic nucleic acid obtained from the cell, tissue, or sample, respectively, after the cell, tissue, or sample, respectively, has been exposed to the agent;
f) either (i) randomly fragmenting the nucleic acid sample or (ii) generating a range of fragments of the nucleic acids from the sample amplified in step e) using one or more restriction enzymes, and then sequencing the resultant fragments;
g) mapping the fragments sequenced in step f) to the reference nucleic acid sequence;
h) comparing the sequences of fragments mapped in step g) to a corresponding portion of the reference nucleic acid sequence so as to identify, and, optionally, quantify, mutation(s), indels and/or genome rearrangements in the genomic nucleic acid of the first sample;
i) comparing the number of mutations, indels and/or genome rearrangements identified or quantified in step h) with the number of mutations, indels and/or genome rearrangements identified or quantified in step d) for one or more sequences, or portion thereof, mapped in both step c) and step g),
wherein an increase in the number of mutations, indels and/or genome rearrangements identified or quantified in step h) compared to step d) in excess of a predetermined control level indicates that the subject is susceptible to the mutagenic agent and wherein the number of mutations, indels and/or genome rearrangements identified or quantified in step h) compared to step d) at or below a predetermined control level indicates that the subject is not susceptible to the mutagenic agent.

A kit is provided comprising reagents and protocol instructions for performing any of the methods disclosed herein.

In an embodiment of the methods the agent is a chemical having a mass of 2000 daltons or less or of 1000 daltons or less. In an embodiment of the methods the chemical is an organic chemical.

In an embodiment of the methods the agent is a radioactive agent. in an embodiment of the methods the agent is a virus. In an embodiment of the methods the agent is a transposon.

In an embodiment of the methods the sample comprises a blood sample. In an embodiment of the methods the sample is a tissue sample. in an embodiment of the methods the sample comprises a cancer cell. In an embodiment of the methods the sample comprises a stem cell.

Also provided is a method for determining if an agent increases somatic mutations in a genome of a cell, tissue or subject exposed to the agent, comprising:

a) accessing, using one or more processors, a first set of data from a database, the first set of data being a reference nucleic acid sequence;
b) mapping to the reference nucleic acid sequence, using one or more processors, sequences of paired-end fragments obtained and sequenced from the genome of the cell, tissue or subject or obtained and sequenced from a nucleic acid amplified from the genome of the cell, tissue or subject, before the genome has been exposed to the agent;
c) comparing, using one or more processors, the sequences of the mapped paired-end fragment sequences to corresponding portions of the reference nucleic acid sequence thereby generating a second set of data which comprises all mutations, indels and/or genome rearrangements identified in the genome of the cell, tissue or subject relative to the reference nucleic acid sequence;
d) accessing, using one or more processors, the first set of data from the database, the first set of data being the reference nucleic acid sequence;
e) after the genome has been exposed to the agent, mapping to the reference nucleic acid sequence, using one or more processors, sequences of paired-end fragments obtained and sequenced from the genome of the cell, tissue or subject or obtained and sequenced from a nucleic acid amplified from the genome of the cell, tissue or subject;
f) comparing, using one or more processors, the sequences of mapped paired-end fragment sequences to corresponding portions of the reference nucleic acid sequence thereby generating a third set of data which comprises mutations, indels and/or genome rearrangements identified in the genome of the cell, tissue or subject, after the genome has been exposed to the agent, relative to the reference nucleic acid sequence; and
g) comparing, using one or more processors, the second set of data and the third set of data, wherein an increase in the number of mutations, indels and/or genome rearrangements identified in the third set of data compared to the second set of data indicates that the agent increases somatic mutations, indels and/or genome rearrangements in the genome of the cell, tissue or subject, respectively, exposed to the agent, and wherein no change or a decrease in the number of mutations, indels and/or genome rearrangements in the third set of data compared to the second set of data indicates that the agent does not increase somatic mutations, indels and/or genome rearrangements in the genome of the cell, tissue, or subject, respectively, exposed to the agent.

Also provided is a system for determining if an agent increases somatic mutations in a genome of a cell, tissue or subject exposed to the agent, comprising:

one or more data processing apparatus; and
a computer-readable medium coupled to the one or more data processing apparatus having instructions stored thereon which, when executed by the one or more data processing apparatus, cause the one or more data processing apparatus to perform a method comprising:
a) accessing, using one or more processors, a first set of data from a database, the first set of data being a reference nucleic acid sequence;
b) mapping to the reference nucleic acid sequence, using one or more processors, sequences of paired-end fragments obtained and sequenced from the genome of the cell, tissue or subject or obtained and sequenced from a nucleic acid amplified from the genome of the cell, tissue or subject, before the genome has been exposed to the agent;
c) comparing, using one or more processors, the sequences of the mapped paired-end fragment sequences to corresponding portions of the reference nucleic acid sequence thereby generating a second set of data which comprises all mutations, indels and/or genome rearrangements identified in the genome of the cell, tissue or subject relative to the reference nucleic acid sequence;
d) accessing, using one or more processors, the first set of data from the database, the first set of data being the reference nucleic acid sequence;
e) after the genome has been exposed to the agent, mapping to the reference nucleic acid sequence, using one or more processors, sequences of paired-end fragments obtained and sequenced from the genome of the cell, tissue or subject or obtained and sequenced from a nucleic acid amplified from the genome of the cell, tissue or subject;
f) comparing, using one or more processors, the sequences of mapped paired-end fragment sequences to corresponding portions of the reference nucleic acid sequence thereby generating a third set of data which comprises mutations, indels and/or genome rearrangements identified in the genome of the cell, tissue or subject, after the genome has been exposed to the agent, relative to the reference nucleic acid sequence; and
g) comparing, using one or more processors, the second set of data and the third set of data, wherein an increase in the number of mutations, indels and/or genome rearrangements identified in the third set of data compared to the second set of data indicates that the agent increases somatic mutations, indels and/or genome rearrangements in the genome of the cell, tissue or subject, respectively, exposed to the agent, and wherein no change or a decrease in the number of mutations, indels and/or genome rearrangements in the third set of data compared to the second set of data indicates that the agent does not increase somatic mutations, indels and/or genome rearrangements in the genome of the cell, tissue, or subject, respectively, exposed to the agent.

Also provided is a computer-readable medium comprising instructions stored thereon which, when executed by a data processing apparatus, causes the data processing apparatus to perform a method comprising:

a) accessing, using one or more processors, a first set of data from a database, the first set of data being a reference nucleic acid sequence;
b) mapping to the reference nucleic acid sequence, using one or more processors, sequences of paired-end fragments obtained and sequenced from the genome of the cell, tissue or subject or obtained and sequenced from a nucleic acid amplified from the genome of the cell, tissue or subject, before the genome has been exposed to the agent;
c) comparing, using one or more processors, the sequences of the mapped paired-end fragment sequences to corresponding portions of the reference nucleic acid sequence thereby generating a second set of data which comprises all mutations, indels and/or genome rearrangements identified in the genome of the cell, tissue or subject relative to the reference nucleic acid sequence;
d) accessing, using one or more processors, the first set of data from the database, the first set of data being the reference nucleic acid sequence;
e) after the genome has been exposed to the agent, mapping to the reference nucleic acid sequence, using one or more processors, sequences of paired-end fragments obtained and sequenced from the genome of the cell, tissue or subject or obtained and sequenced from a nucleic acid amplified from the genome of the cell, tissue or subject;
f) comparing, using one or more processors, the sequences of mapped paired-end fragment sequences to corresponding portions of the reference nucleic acid sequence thereby generating a third set of data which comprises mutations, indels and/or genome rearrangements identified in the genome of the cell, tissue or subject, after the genome has been exposed to the agent, relative to the reference nucleic acid sequence; and
g) comparing, using one or more processors, the second set of data and the third set of data, wherein an increase in the number of mutations, indels and/or genome rearrangements identified in the third set of data compared to the second set of data indicates that the agent increases somatic mutations, indels and/or genome rearrangements in the genome of the cell, tissue or subject, respectively, exposed to the agent, and wherein no change or a decrease in the number of mutations, indels and/or genome rearrangements in the third set of data compared to the second set of data indicates that the agent does not increase somatic mutations, indels and/or genome rearrangements in the genome of the cell, tissue, or subject, respectively, exposed to the agent.

Also provided is a method for determining if an agent increases somatic mutations in a genome of a cell, tissue or subject exposed to the agent, comprising:

a) accessing, using one or more processors, a first set of data from a database, the first set of data being a reference nucleic acid sequence;
b) mapping to the reference nucleic acid sequence, using one or more processors, sequences of fragments obtained and sequenced from the genome of the cell, tissue or subject or obtained and sequenced from a nucleic acid amplified from the genome of the cell, tissue or subject, before the genome has been exposed to the agent;
c) comparing, using one or more processors, the sequences of the mapped fragment sequences to corresponding portions of the reference nucleic acid sequence thereby generating a second set of data which comprises all mutations, indels and/or genome rearrangements identified in the genome of the cell, tissue or subject relative to the reference nucleic acid sequence;
d) accessing, using one or more processors, the first set of data from the database, the first set of data being the reference nucleic acid sequence;
e) after the genome has been exposed to the agent, mapping to the reference nucleic acid sequence, using one or more processors, sequences of fragments obtained and sequenced from the genome of the cell, tissue or subject or obtained and sequenced from a nucleic acid amplified from the genome of the cell, tissue or subject;
f) comparing, using one or more processors, the sequences of mapped fragment sequences to corresponding portions of the reference nucleic acid sequence thereby generating a third set of data which comprises mutations, indels and/or genome rearrangements identified in the genome of the cell, tissue or subject, after the genome has been exposed to the agent, relative to the reference nucleic acid sequence; and
g) comparing, using one or more processors, the second set of data and the third set of data, wherein an increase in the number of mutations, indels and/or genome rearrangements identified in the third set of data compared to the second set of data indicates that the agent increases somatic mutations, indels and/or genome rearrangements in the genome of the cell, tissue or subject, respectively, exposed to the agent, and wherein no change or a decrease in the number of mutations, indels and/or genome rearrangements in the third set of data compared to the second set of data indicates that the agent does not increase somatic mutations, indels and/or genome rearrangements in the genome of the cell, tissue, or subject, respectively, exposed to the agent.

Also provided is a system for determining if an agent increases somatic mutations in a genome of a cell, tissue or subject exposed to the agent, comprising: one or more data processing apparatus; and

a computer-readable medium coupled to the one or more data processing apparatus having instructions stored thereon which, when executed by the one or more data processing apparatus, cause the one or more data processing apparatus to perform a method comprising:
a) accessing, using one or more processors, a first set of data from a database, the first set of data being a reference nucleic acid sequence;
b) mapping to the reference nucleic acid sequence, using one or more processors, sequences of fragments obtained and sequenced from the genome of the cell, tissue or subject or obtained and sequenced from a nucleic acid amplified from the genome of the cell, tissue or subject, before the genome has been exposed to the agent;
c) comparing, using one or more processors, the sequences of the mapped fragment sequences to corresponding portions of the reference nucleic acid sequence thereby generating a second set of data which comprises all mutations, indels and/or genome rearrangements identified in the genome of the cell, tissue or subject relative to the reference nucleic acid sequence;
d) accessing, using one or more processors, the first set of data from the database, the first set of data being the reference nucleic acid sequence;
e) after the genome has been exposed to the agent, mapping to the reference nucleic acid sequence, using one or more processors, sequences of fragments obtained and sequenced from the genome of the cell, tissue or subject or obtained and sequenced from a nucleic acid amplified from the genome of the cell, tissue or subject;
f) comparing, using one or more processors, the sequences of mapped fragment sequences to corresponding portions of the reference nucleic acid sequence thereby generating a third set of data which comprises mutations, indels and/or genome rearrangements identified in the genome of the cell, tissue or subject, after the genome has been exposed to the agent, relative to the reference nucleic acid sequence; and
g) comparing, using one or more processors, the second set of data and the third set of data, wherein an increase in the number of mutations, indels and/or genome rearrangements identified in the third set of data compared to the second set of data indicates that the agent increases somatic mutations, indels and/or genome rearrangements in the genome of the cell, tissue or subject, respectively, exposed to the agent, and wherein no change or a decrease in the number of mutations, indels and/or genome rearrangements in the third set of data compared to the second set of data indicates that the agent does not increase somatic mutations, indels and/or genome rearrangements in the genome of the cell, tissue, or subject, respectively, exposed to the agent.

A computer-readable medium comprising instructions stored thereon which, when executed by a data processing apparatus, causes the data processing apparatus to perform a method comprising:

a) accessing, using one or more processors, a first set of data from a database, the first set of data being a reference nucleic acid sequence;
b) mapping to the reference nucleic acid sequence, using one or more processors, sequences of fragments obtained and sequenced from the genome of the cell, tissue or subject or obtained and sequenced from a nucleic acid amplified from the genome of the cell, tissue or subject, before the genome has been exposed to the agent;
c) comparing, using one or more processors, the sequences of the mapped fragment sequences to corresponding portions of the reference nucleic acid sequence thereby generating a second set of data which comprises all mutations, indels and/or genome rearrangements identified in the genome of the cell, tissue or subject relative to the reference nucleic acid sequence;
d) accessing, using one or more processors, the first set of data from the database, the first set of data being the reference nucleic acid sequence;
e) after the genome has been exposed to the agent, mapping to the reference nucleic acid sequence, using one or more processors, sequences of fragments obtained and sequenced from the genome of the cell, tissue or subject or obtained and sequenced from a nucleic acid amplified from the genome of the cell, tissue or subject;
f) comparing, using one or more processors, the sequences of mapped fragment sequences to corresponding portions of the reference nucleic acid sequence thereby generating a third set of data which comprises mutations, indels and/or genome rearrangements identified in the genome of the cell, tissue or subject, after the genome has been exposed to the agent, relative to the reference nucleic acid sequence; and
g) comparing, using one or more processors, the second set of data and the third set of data, wherein an increase in the number of mutations, indels and/or genome rearrangements identified in the third set of data compared to the second set of data indicates that the agent increases somatic mutations, indels and/or genome rearrangements in the genome of the cell, tissue or subject, respectively, exposed to the agent, and wherein no change or a decrease in the number of mutations, indels and/or genome rearrangements in the third set of data compared to the second set of data indicates that the agent does not increase somatic mutations, indels and/or genome rearrangements in the genome of the cell, tissue, or subject, respectively, exposed to the agent.

In an embodiment of the instant method, system or computer-readable medium, the fragments sequenced in steps b) and/or e) are sequenced by paired-end sequencing.

In an embodiment of the methods the fragments represent up to 2% of the genome. In an embodiment of the methods the fragments represent up to 1% of the genome.

In an embodiment the methods further comprise obtaining the sample of the nucleic acid from the subject prior to step a).

Also provided is an apparatus system for determining if an agent increases somatic mutations in a genome of a cell, tissue or subject exposed to the agent, comprising: one or nucleic acid more sequencing machine(s) and, optionally, one or more data processing apparatus and a computer-readable medium coupled to the one or more data processing apparatus having instructions stored thereon which, when executed by the one or more data processing apparatus, cause the one or more data processing apparatus to perform a method comprising:

a) accessing, using one or more processors, a first set of data from a database, the first set of data being a reference nucleic acid sequence;
b) mapping to the reference nucleic acid sequence, using one or more processors, sequences of fragments obtained and sequenced from the genome of the cell, tissue or subject or obtained and sequenced from a nucleic acid amplified from the genome of the cell, tissue or subject, before the genome has been exposed to the agent, wherein the fragments are sequenced by the one or more sequencing machine(s);
c) comparing, using one or more processors, the sequences of the mapped fragment sequences to corresponding portions of the reference nucleic acid sequence thereby generating a second set of data which comprises all mutations, indels and/or genome rearrangements identified in the genome of the cell, tissue or subject relative to the reference nucleic acid sequence;
d) accessing, using one or more processors, the first set of data from the database, the first set of data being the reference nucleic acid sequence;
e) after the genome has been exposed to the agent, mapping to the reference nucleic acid sequence, using one or more processors, sequences of fragments obtained and sequenced from the genome of the cell, tissue or subject or obtained and sequenced from a nucleic acid amplified from the genome of the cell, tissue or subject, wherein the fragments are sequenced by the one or more sequencing machine(s);
f) comparing, using one or more processors, the sequences of mapped fragment sequences to corresponding portions of the reference nucleic acid sequence thereby generating a third set of data which comprises mutations, indels and/or genome rearrangements identified in the genome of the cell, tissue or subject, after the genome has been exposed to the agent, relative to the reference nucleic acid sequence; and
g) comparing, using one or more processors, the second set of data and the third set of data, wherein an increase in the number of mutations, indels and/or genome rearrangements identified in the third set of data compared to the second set of data indicates that the agent increases somatic mutations, indels and/or genome rearrangements in the genome of the cell, tissue or subject, respectively, exposed to the agent, and wherein no change or a decrease in the number of mutations, indels and/or genome rearrangements in the third set of data compared to the second set of data indicates that the agent does not increase somatic mutations, indels and/or genome rearrangements in the genome of the cell, tissue, or subject, respectively, exposed to the agent. “Sequencing machine(s)” as used herein encompasses automatic sequencers as available in the art.

A kit is provided comprising reagents and protocol instructions for performing one of the instant methods.

In an embodiment, somatic point mutations and germline variation can be scored using a SAMtools (Li, H., Handsaker, B., Wysoker, A., Fennell, T., Ruan, J., Horner, N., Marth, G., Abecasis, G. and Durbin, R. (2009) The Sequence Alignment/Map format and SAMtools. Bioinformatics, 25, 2078-2079, hereby incorporated by reference) and by VarScan somatic command. (Koboldt, D. C., Chen, K., Wylie, T., Larson, D. E., McLellan, M. D., Mardis, E. R., Weinstock, G. M., Wilson, R. K. and Ding, L. (2009) VarScan: variant detection in massively parallel sequencing of individual and pooled samples. Bioinformatics, 25, 2283-2285, hereby incorporated by reference). A minimum base quality score of 10, 15, 20, 25, 30 35 or 40 and a minimum mapping quality score of 10, 15, 20, 25, 30 35 or 40 can be set in the VarScan command. In a preferred embodiment, a minimum base quality score of 20 is set. In a preferred embodiment, the minimum mapping quality score is 20. In embodiments, the minimum read depth is 10, 15, 20, 25, 30 35 or 40 for either or both the unamplified sample and the single cell. In a preferred embodiment, the minimum read depth is 10. In embodiments, the minimum mutant allele frequency is 10%, 15%, 20%, 25%, 30%, 35%, 40% or 45% for point mutations found in the single cell. In a preferred embodiment, the minimum mutant allele frequency is 20% for point mutations found in the single cell. In an embodiment, a strand bias script can be used to filter out events where the variant allele is biased towards reads aligning to one strand.

In an embodiment, filtered somatic point mutations can be visually validated using a an appropriate batch script that records images of aligned reads at each locus containing a somatic point mutation (for example, see Robinson, J. T., Thorvaldsdottir, H., Winckler, W., Guttman, M., Lander, E. S., Getz, G. and Mesirov, J. P. (2011) Integrative genomics viewer. Nat. Biotechnol., 29, 24-26, hereby incorporated by reference). Select point mutations were chosen for further validation using Sanger sequencing. Primers were designed to flank either side of the mutant of interest. DNA from the single cell containing the somatic mutation and the cell line were tested and the trace images were inspected to confirm that the wild type and mutant alleles (trace peaks) were found at the expected ratio.

As used herein, the polymerase chain reaction (“PCR”) is a technique well-known in the art to amplify a single or a few copies of a piece of DNA across several orders of magnitude by use of thermal cycling, consisting of cycles of repeated heating arid cooling of the reaction for DNA melting and enzymatic replication of the DNA and primers containing sequences complementary to the target region along with a DNA polymerase (for example, see PCR Primer: A Laboratory Manual, Second Edition, edited by Carl W. Dieffenbach and Gabriela S. Dveksler, Cold Spring Harbor Laboratory Press, 2003, ISBN 978-087969654-2, which is hereby incorporated by reference).

Sequencing of a nucleic acid, as the term is used herein, can be by any method known in the art including, but not limited to, sequencing-by-synthesis methods, including chain termination methods, ligation-mediated sequencing methods, single-molecule sequencing methods, nanopore sequencing methods and semi-conductor-based sequencing methods. In embodiments the fragments are 25-50 base pairs (bp), 50-100 bp, 100-200 bp, 200-300 bp, 300-400 bp, 400-500 bp, 500-600 bp, 600-700 bp, 700-800 bp, 800-900 bp, 1000-2000 bp, 2000-3000 bp, 3000-4000 bp, 4000-5000 bp, 5000-6000 bp, 6000-7000 bp, 7000-8000 bp, 8000-9000 bp, 9000-10,000 bp, 10,000-20,000 bp, 20,000-30,000 bp, 30,000-40,000 bp, 40,000-50,000 bp, 50,000-60,000 bp, 60,000-70,000 bp, 70,000-80,000 bp, 80,000-90,000 bp, 90,000-100,000 bp, 100,000-200,000 bp, or up to 250,000 bp. Size-selection of fragments by any technique known in the art, including but not limited to agarose gel selection, can be used to select out any desired fragment size or range of fragment sizes.

In an embodiment of the methods, sequence (e.g. genome) rearrangement artifacts are accounted for by removing identified rearrangements (for example, by identification through paired end sequencing) from the sequencing results. in an embodiment of the methods, DNA mutation load at any desired locus can be derived computationally as the ratio of (sequence variants) versus (the total number of wild type sequences minus the artificially-induced mutant fragments).

The methods disclosed herein can be applied, mutatis mutandis, to the transcriptome, but the mRNA must be converted into cDNA, which is then subjected to the methods described herein.

As used herein “mapping” means, in regard to a first nucleic acid sequence and a reference nucleic acid sequence, locating on the reference nucleic acid sequence the position to which the first sequence nucleic acid corresponds. Paired-end sequencing is particularly assistive for such mapping. A paired-end sequencing strategy enables robust mapping and characterization of fragments, and thereby, the original sample. Point mutations are readily identified, as are deletions and insertions compared to the reference in view of the fragment length. Mapping to the reference sequence can be at 70-95% identity, 95% or greater identity, 96% or greater identity, 97% or greater identity, 98% or greater identity, 95% or greater identity, or 100% identity.

As used herein “amplifying” a given nucleic acid means increasing the copy number of that nucleic acid by, e.g., any of the standard techniques for amplifying nucleic acids known in the art.

As used herein, a “restriction enzyme” is a restriction endonuclease that cuts double-stranded or single stranded DNA at specific recognition nucleotide sequences known as restriction sites. Restriction enzymes are well-known in the art. in an embodiment, the restriction enzyme is a 4-base cutter. In an embodiment, the restriction enzyme is HindIII, PstI or MseI.

As used herein a “reference nucleic acid sequence” is a nucleic acid sequence which is used as a standard for mapping and comparing other sequences to, for purposes of identifying differences. For example, a reference nucleic acid sequence, usually predetermined, may be obtained from a database available in the art, e.g. RefSeq as supplied at www.ncbi.nlm.nih.gov/RefSeq/, or obtained by sequencing a nucleic acid from, for example, other members, including a plurality of, the cell, tissue or subject population on which the method is being applied. In one embodiment, the reference sequence is the human genome hg19. In an embodiment, the reference nucleic acid sequence is the wildtype nucleic acid sequence.

As used herein a “corresponding portion” of a reference nucleic sequence is a portion of the reference nucleic sequence that aligns with or matches, as determined for example by sequence alignment/map tools widely available in the art, the sequence being compared.

Embodiments of the invention and all of the functional operations described in this specification can be implemented in digital electronic circuitry, or in computer software, firmware, or hardware, including the structures disclosed in this specification and their structural equivalents, or in combinations of one or more of them. Embodiments of the invention can be implemented as one or more computer program products, i.e., one or more modules of computer program instructions encoded on a computer readable medium for execution by, or to control the operation of, data processing apparatus. The computer readable medium can be a machine readable storage device, a machine readable storage substrate, a memory device, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the invention can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the invention can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

Where a numerical range is provided herein, it is understood that all numerical subsets of that range, and all the individual integers contained therein, are provided as part of the invention. Thus, a fragment which is from 25 to 50 base pairs in length includes the subset of fragments which are 25 to 30 base pairs in length, the subset of fragments which are 35 to 42 base pairs in length etc. as well as a fragment which is 25 base pairs in length, a fragment which is 26 base pairs in length, a fragment which is 27 base pairs in length, etc. up to and including a fragment which is 50 base pairs in length.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Introduction

DNA mutations are the inevitable consequences of errors that arise during replication and repair of DNA damage. Because of their random and infrequent occurrence, quantification and characterization of DNA mutations in the genome of somatic cells of multicellular organisms has been difficult. Current estimates of somatic mutation rates in metazoa are based on selectable reporter loci (1), which are unlikely to be representative for the genome overall. With the emergence of massively parallel sequencing (MPS) it has now become possible to comprehensively analyze whole genomes for all possible mutations, but only in clonally-derived genomic DNA. For example, the 1000 Genomes Project (2,3) detects mutations as genetic variants among individuals, and the Cancer (Genome Atlas (4) catalogues mutations in clonally derived tumor tissues. To account for sequencing errors current MPS protocols for mutation detection are based on a consensus model, i.e., finding the same event in multiple, independent reads from the same locus. This allows only the detection of clonally amplified mutations present in most or all of the cells in a tissue sample and essentially constrains access to the far majority of all mutations, which are present only in a small fraction of all cells and cannot be distinguished from sequencing errors. One way to circumvent this problem and account for the mutational heterogeneity within tissues is whole genome sequencing of a representative number of single cells. However, there are basically two factors that effectively constrain direct measurements of somatic mutations, which are unique events and may be found only once among many different sequencing reads. Firstly, how does one obtain high enough coverage to detect low-frequency somatic mutations without dramatically increasing the cost of sequencing? And, secondly, how to distinguish those sequence variants that are merely artifacts of the procedure and those that are truly unique mutations? The present invention can address both problems simultaneously.

Here it is shown that significantly elevated mutation loads presented as genome-wide mutation frequencies and spectra in single cells from Drosophila melanogaster S2 and mouse embryonic fibroblast (MEF) populations after treatment with the powerful mutagen N-ethyl-N-nitrosourea (ENJ). This first direct assessment of mutagenic effects across single cells allows tracking cell-to-cell variability in mutagenic effects on tissues and provide insight into the pathogenic history of disease-causing mutations and the mechanisms of their induction. Importantly, it provides the first direct measure for estimating cancer risk in subjects exposed to environmental mutagens, such as radiation.

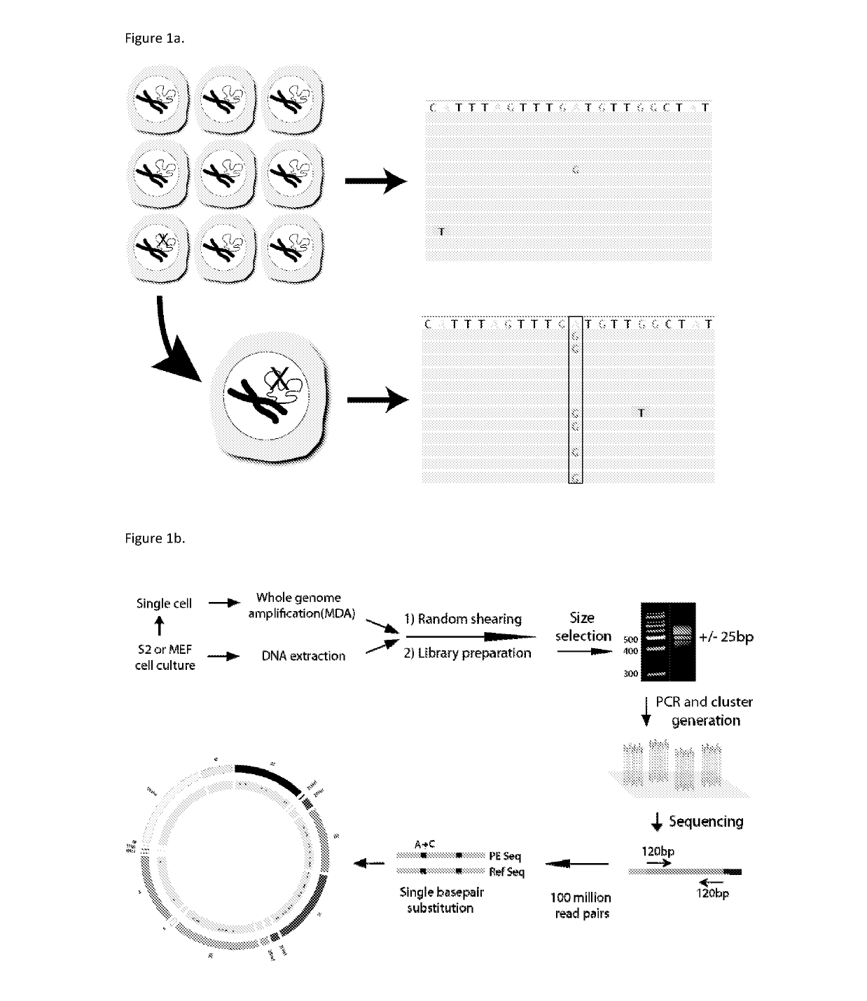

DNA mutation is the ultimate source of genetic variation, both adaptive and deleterious. With the emergence of massively parallel sequencing (MPS) there is now access to germline DNA mutations or clonally amplified mutations in tumors. What has not been possible, however, is the assessment of somatic mutation frequencies and spectra across the genome in somatic cell populations. This is due to the relatively high error rate of current MPS platforms, which is in the order of 1-10×10⁻³ (5). This prevents one from simply sequencing across a locus a large number of times and counting the number of mutant reads. A mutation derived from one particular cell in a tissue cannot be distinguished from a sequencing error. One way to circumvent this problem is to sequence the genomes of individual cells after whole genome amplification. Every mutation in that cell at a particular locus will then act as the consensus sequence (FIG. 1A).

To obtain high coverage it is preferred to selectively target certain regions of the genome and sequence those preferentially. To eliminate artifacts from real mutations, the signature of the artifacts is defined and filtered out through the use of, for example, an in silico filtering algorithm. Samples can be fragmented/prepared by restriction digestion, and subsequent selection of a particular size range of fragments representing, e.g., approximately 1% of the genome. This generates a library containing fragments of known size and known genomic coordinates. Over 99% of the fragments in this library correctly map to the genomic coordinates expected. This procedure gives 10-100.000-fold coverage, depending on the genome size and allows a representative estimate of the DNA mutation content of the genome.

For genome rearrangements, the formation of chimeric artifacts during the library preparation and sequencing is due to random fragments present in the library “crossing over”. This leads to the first of two paired-reads representing one fragment and the second read representing a different fragment. By defining the set of fragments present in the library through the restriction digestion one can filter out chimeras that occur between any two of these fragments. This filtering only removes 0.5% of true positives, while removing over 99.99% of false positives. The technique's high rate of filtering out false positives enables the accurate estimation of translocation frequencies of as low as 1 in 10 million reads. In an embodiment of the methods, genome rearrangement artifacts are accounted for by removing identified rearrangements (for example, by identification through paired end sequencing) from the sequencing results.

Accordingly, this invention relates to a method for measuring genetic or epigenetic DNA mutational profiles in primary cells or tissues of subjects such as plants, animals or humans. The method can use, in embodiments, genomic DNA fragments obtained by (1) restriction enzyme digestion; (2) whole genome amplification of small DNA samples down to single genomes; or (3) a combination of the two, to prepare a library for paired-end DNA sequencing. DNA mutation load at any desired locus can be derived computationally as the ratio of sequence variants versus the total number of wild type sequences minus the artificially-induced mutant fragments, which are filtered out.

### Results

Genomic DNA from cultured mouse (embryonic fibroblasts; MEFs) and fly (Drosophila melanogaster embryo cell line; S2) cells was analyzed by paired-end (“PE”) sequencing after a restriction enzyme digestion (HindIII for mouse and PstI for fly) and size selection. The fly and mouse genomes are structured very differently, with the mouse genome consisting of close to 50% repetitive DNA and the fly genome only 3%. The libraries were sequenced using a paired-end kit on the Illumina® Genome Analyzer fix with a read length of 85 bp. The paired-end sequences were aligned to a reference genome sequence (RefSeq; Mouse Build 37.1 or Drosophila DM3) using the Burrows-Wheeler Aligner (BWA) (e.g. available at bio-bwa.sourceforge.net) and then sorted/indexed using the Sequence Alignment/Map tools (SAMtools). Alterations in the distance between two PE reads relative to the distance predicted by the RefSeq indicate putative genome rearrangements. Artifacts were eliminated in the following way. First, any read pairs that had mapping quality scores lower than 30 (e.g. see Li H, Ruan J, Durbin R. (2008) Mapping short DNA sequencing reads and calling variants using mapping quality scores. Genome Research 18:1851-1858, hereby incorporated by reference in its entirety, for mapping scores) were filtered out. This removed repeat-spanning reads with ambiguous placement on the reference genome. Then a script was implemented to remove chimeric sequences. This script used a hash table with the genome coordinates of each restriction site, and the resulting fragment sizes following digestion, to qualify rearrangements as chimeras or non-chimeras. Statistical modeling showed that this filtering algorithm removes 0.5% of true positives, while removing over 99.99% of false positives.

It was reasoned that treatment of S2 cells with a clastogen should give rise to elevated frequencies of genome rearrangements. Moreover, a direct comparison between mouse and fly cells should result in a significantly higher frequency of genome rearrangements in the invertebrate species. Results were obtained from the reduced representation assay with MEFs and S2 cells, with the S2 cells before and after treatment with the powerful clastogen bleomycin. The frequencies of genome rearrangements were expressed per read pair. When comparing fly with mouse cells an approximately 5-fold higher frequency of rearrangements in fly cells was noted. Taking into account that the target size of the lacZ gene is an order of magnitude greater that the MPS target size (350-550 bp, see above), this is roughly similar to a previous result in this laboratory using the lacZ reporter gene in which, for the first time, spontaneous mutations in these two species was compared. Also observed was an approximately 3-fold increase in genomic mutation frequency in bleomycin-treated S2 cells as compared to untreated cells.

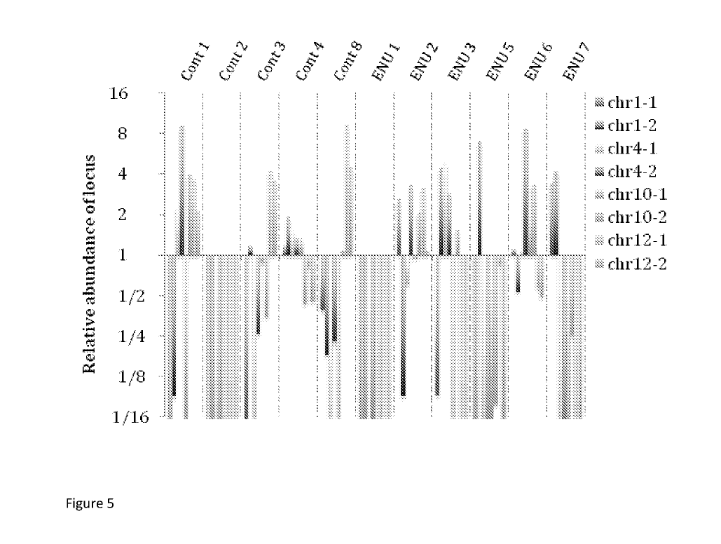

To assess somatic mutation spectra in single cells in a genome-wide manner S2 cells derived from Drosophila melanogaster, an organism with a genome size of 160 MB, were used. The strategy followed is outlined in FIG. 1B. Individual single cells were picked from an S2 cell culture 72-h after treatment with 4.2 mM of the powerful point mutagen N-ethyl-N-nitrosourea (ENU) or mock treated with solvent (control). At 72-h post-treatment virtually no lesions remain (6,7) and cell survival is greater than 90% (8) (results not shown). Each single cell was lysed and subjected to whole genome amplification (WGA) using an optimized multiple displacement amplification (MDA) protocol (see Methods and Materials)(9). The amplified cells were first screened for locus dropout by qPCR using primer pairs distributed over the different chromosomes (FIG. 5). DNA from the cells displaying the least amount of dropout was fragmented and processed to generate sequencing libraries for the Illumina HiSeq platform. In this way libraries of three untreated and three ENU-treated cells were prepared. For comparison, a similar library was made from unamplified total genomic DNA from the untreated S2 cell population. To identify all possible mutations, either spontaneously formed in the unexposed, control cells or induced by ENU in the treated cells, the libraries were sequenced from both ends, generating between 50-100 million 108-bp paired-end reads per cell. Alignment to the Drosophila reference sequence (dm3) was performed using BWA (10), and post-processing was completed using the Genome Analysis Toolkit (GATK) (11). Variant analysis revealed point mutations, small indels and genome rearrangements. Since ENU is a point mutagen the subsequent analysis was based on this type of mutations only. The pipeline developed for somatic point mutation discovery is described in further detail in the Methods and Materials section. Briefly, aligned sequence data from the unamplified sample and a single cell was compared and all differences with the reference sequence were recorded as variants. Variants with sufficient coverage (20×) in both the unamplified and the single cell sample were classified as “germline” or “somatic” based on whether the variant was shared between the two samples. Somatic variants were further processed using a strand-bias filter and were visually validated using the Integrative Genomics Viewer (IGV) (12).

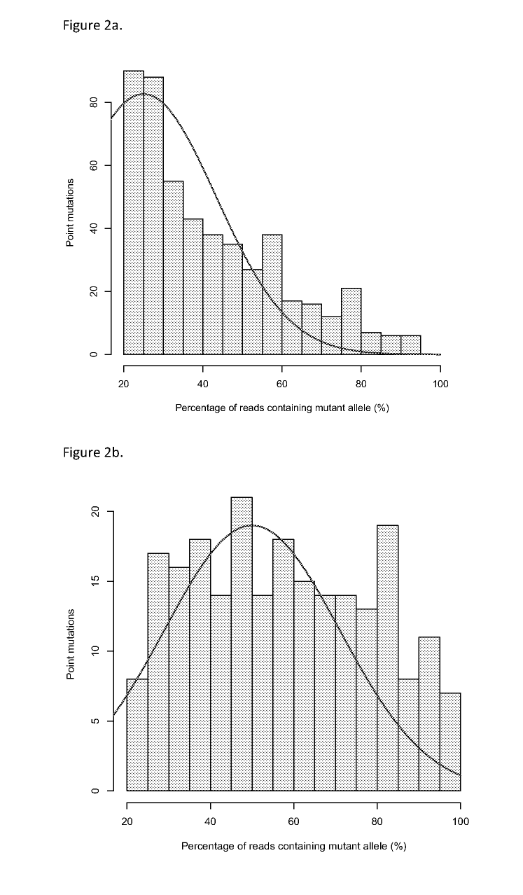

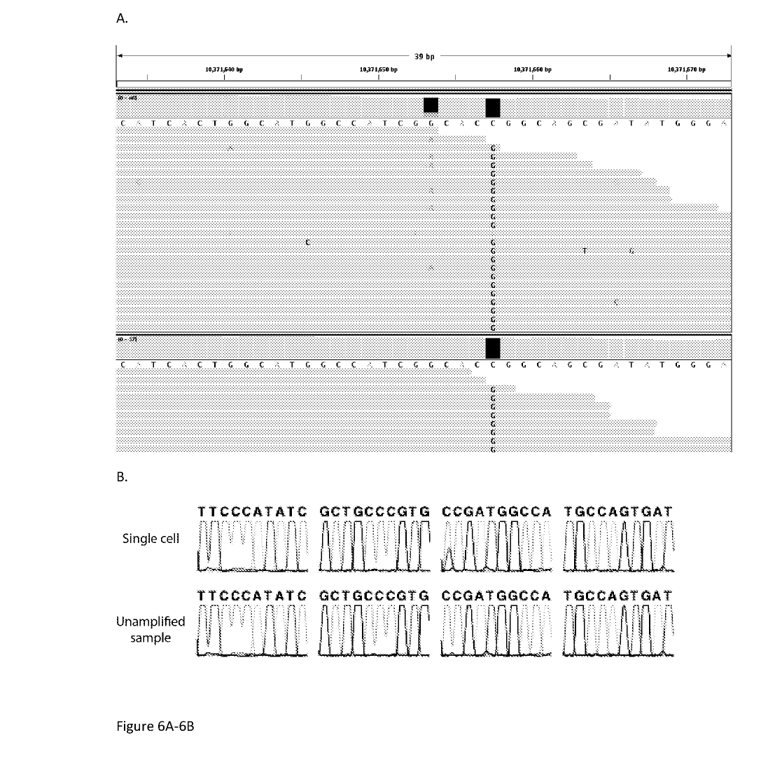

The results indicate sufficient coverage (20×) for genotyping at between 40% and 80% of bases in the genome (Table 1). The incomplete coverage is due to amplification bias, which can be pronounced especially with small template amounts (9). While the WGA protocol was optimized, locus dropout was still observed, as was a significant level of allele dropout. The latter was measured using both heterozygous SNPs present in the S2 cell line population and the mutant read frequency distribution, which produced similar results. Approximately 500,000 polymorphic differences with the reference genome were detected in the unamplified cell line DNA in the form of single base SNPs, indels, and CNVs. FIG. 2 shows a Circos plot of the somatic point mutations (interior) in the ENU- and control S2 single-cell genomes, along with a coverage track (exterior) with an upper limit of 50×. These results indicate a 7.5-fold induction of point mutations by ENU on average in the exposed cells. Multiple somatic mutations were chosen for validation with Sanger sequencing using the remaining amplified material from the single cells and no evidence of false positives was found (FIG. 6).

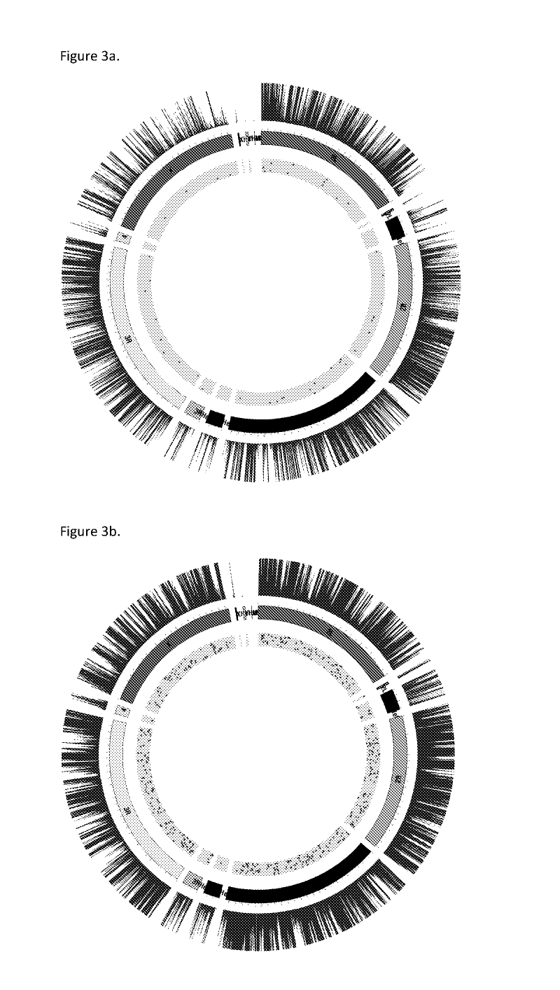

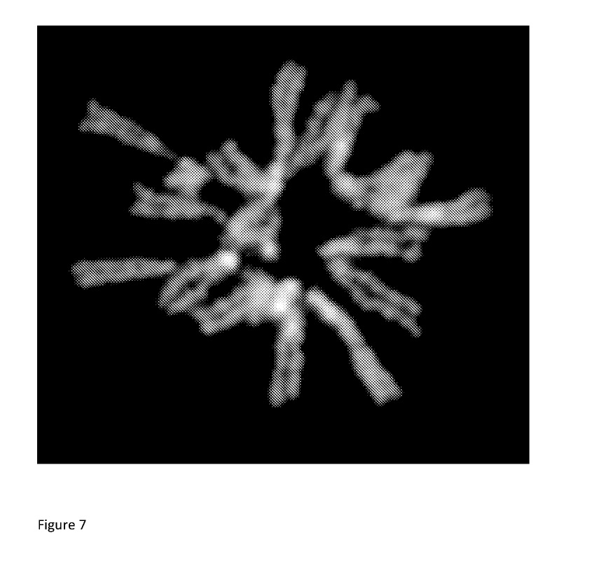

While spontaneous mutations present in the untreated cells could be expected to occasionally be homozygous, all ENU-induced mutations are likely to involve one allele only. Since the S2 cell line is known to be tetraploid (13) (FIG. 7) this can readily be tested. Assuming an equal representation of each allele in the whole genome amplified material from the single cells, one would expect a quarter of the reads aligning across a spontaneous or induced mutation to contain the mutant base. FIG. 3A shows the mutant read frequencies across chr2L for the ENU-induced mutations. While the expected read frequency of 25% was found for chr2L, the significant tail to higher frequencies indicates the unequal amplification across the four alleles. Since the S2 cell line is male, there are two X chromosomes in addition to the four sets of autosomes. Hence, one would expect a read frequency peak at 50% rather than 25% for chrX and this is indeed what was found (FIG. 3B).

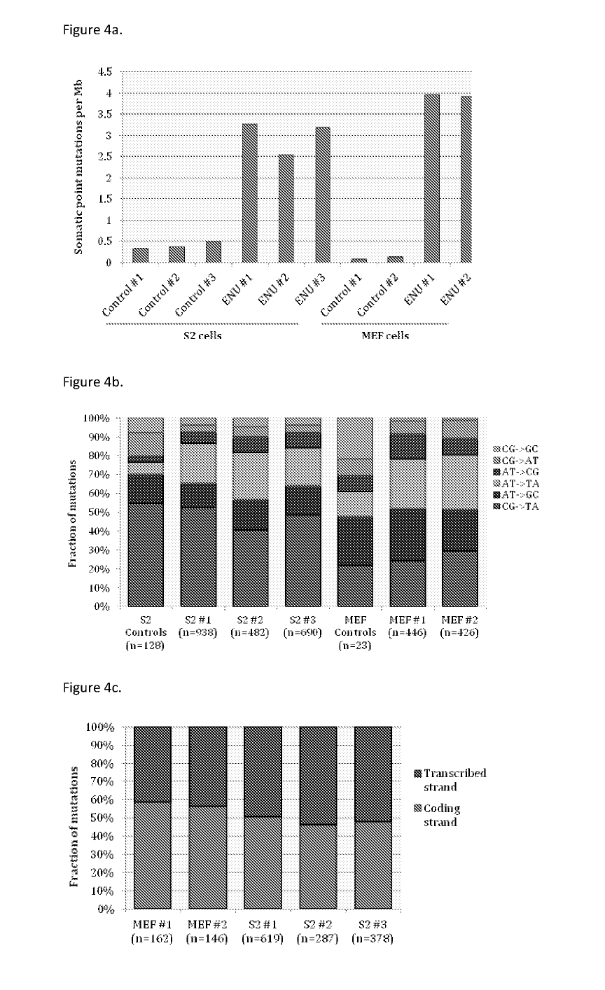

To apply the same strategy to mammalian cells is significantly more expensive because of the much larger genome size. Therefore, the procedure shown in FIG. 1B was slightly modified, using a reduced representation approach based on restriction enzyme digestion. For this purpose mouse embryonic fibroblast (MEF) populations were used either treated with 4.2 mM ENU or mock-treated with solvent only. Instead of preparing sequencing libraries directly using randomly fragmented DNA, whole genome amplified DNA was digested from two treated and two control MEFs, as well as unamplified genomic DNA from the MEF population, with MseI, a four-base cutter with a TTAA cleavage site. Following digestion an agarose gel size-selection was performed for fragments between 250-bp and 350-bp, corresponding to a target region of approximately 300 MB. The fragments were sequenced using 121-bp single-end reads. Alignment to the Mus musculus reference sequence (mm9) and implementation of a variant analysis pipeline revealed a significant number of point mutations induced by ENU in the two cells from the exposed population, similar to what was observed for the S2 cells (FIG. 4A). Due to the nature of the reduced representation library, the strand bias filter could not be used and therefore a more stringent mutant read frequency cutoff (>40%) was adopted. Out of the 300-MB target region, 220 MB (73%) had sufficient coverage (10×) in the unamplified control sample and 85-93 MB (39-42%) of the 220 MB overlapped with regions of sufficient coverage in the single MEFs. Due to the absence of a sufficient number of heterozygous SNPs in the MEF cell line, allele dropout was estimated using the distribution of mutant read frequencies found in the ENU-treated MEF cells. The results indicate a 35-fold induction of point mutations in the ENU-treated MEF cells (FIG. 4A). Previously, using a lacZ reporter in the same cell population, a significantly smaller number of ENU-induced mutations was observed (8), underscoring the reduced sensitivity of reporter systems, which can only detect mutations that alter the phenotype to a considerable extent (1).

The much higher fold induction of mutations in the ENU-treated MEFs than in the S2 cells of the fly is almost entirely due to a lower baseline mutation frequency in the two untreated MEFs. This is not surprising since the S2 cell line used has a long history of passaging during which mutations are likely to accumulate. Indeed, a number of heterozygous SNPs were observed in this cell line, but not in the MEFs. It has previously been demonstrated, using the lacZ reporter locus in MEFs, that during passaging point mutations also accumulate in these cells (14). Baseline levels of somatic mutation frequencies are obviously very difficult to determine with high accuracy and in this case also depend on the cut-offs used to filter out potential artifacts. Here, comparing the absolute number of mutations per MB induced by ENU in cells from the two species was investigated, which proved remarkably similar. Indeed, the ENU-induced mutation frequency in the MEF cells was only 30% higher than that found in the S2 cells (FIG. 4A). Somatic mutation rates are a measure for the efficiency of an organism to cope with DNA damage and it is somewhat surprising that cells from such disparate species are very similar in this respect.

A major advantage of direct sequencing is that the mutation spectra can immediately be visualized across the genome. The majority of ENU-induced DNA damage occurs in the form of nitrogen alkylation and can be repaired in both flies and mice by nucleotide excision repair (NER) (15, 16), which is error-free (17). Oxygen alkylation, on the other hand, positively correlates with the induction of point mutations through the formation of O2-ethyl-thymine, O4-ethyl-thymine, and O6-ethyl-guanine adducts, as well as other minor adducts (18). These adducts tend to cause T->A, T->C and G->A mutations, respectively, which represented the majority of the ENU-induced mutations observed in the S2 and MEF cells (FIG. 4B). The ENU-induced spectrum was highly consistent across individual cells from the same population, but a larger fraction of C:G->T:A mutations was found in the S2 cells. This may be due to the increased repair of O6-ethyl-guanine adducts by the mouse O-6-methylguanine-DNA methyltransferase gene (Mgmt) compared with the Drosophila homologue (19). In spite of this difference, the similarity between the two species also at this level is striking. The spontaneous mutation spectra observed in the untreated cells were similar to the ENU-induced spectra except for the fraction of A:T->T:A mutations. These transversions are known to be highly enriched following treatment with alkylating agents (18, 20-22). In general both the ENU-induced and spontaneous mutations in the MEF cells were predominantly found at A:T bases, whereas the majority of mutations occurred at C:G bases in both the untreated and ENU-treated S2 cells.

Since ENU is a small, direct acting agent, a large bias for mutations localized in accessible or euchromatic regions of the genome was not expected. By comparing data on the accessibility of the S2 cell line (23) with the coordinates of the point mutations it was determined that there was no correlation between mutation localization and genome accessibility. There was also no apparent correlation between a functional category (exon, intron, or intergenic region) and frequencies of mutations for either the ENU-induced or spontaneous mutations found in the two cell populations (Table 1).

TABLE 1
Single cell sequencing data

| | | | | Fraction | | |
|---|---|---|---|---|---|---|
| | Point | Bases in genome | | of genome | Alleles | Muta- |
| Single | muta- | with sufficient | | target | repre- | tions per |
| cell | tions | coverage | | region | sented * | Mb |
| S2 Cont. 1 | 45 | 58.97 | Mb | 50.56% | 56.68% | 0.34 |
| S2 Cont. 2 | 43 | 53. | Mb | 45.44% | 55.95% | 0.36 |
| S2 Cont. 3 | 40 | 37.17 | Mb | 31.87% | 54.33% | 0.50 |
| S2 ENU 1 | 938 | 97.74 | Mb | 83.80% | 73.36% | 3.27 |
| S2 ENU 2 | 482 | 82.58 | Mb | 70.80% | 57.44% | 2.54 |
| S2 ENU 3 | 690 | 90.05 | Mb | 77.16% | 60.27% | 3.18 |
| MEF Cont. 1 | 9 | 85.17 | Mb | 38.71% | ~60% | 0.09 |
| MEF Cont. 2 | 14 | 89.42 | Mb | 40.65% | ~60% | 0.13 |
| MEF ENU 1 | 426 | 89.69 | Mb | 40.77% | 59.89% | 3.97 |
| MEF ENU 2 | 446 | 92.98 | Mb | 42.27% | 61.34% | 3.91 |

Nor was there a correlation between proximity to a replication origin (24) and mutation frequency. Analysis of the ENU-induced mutations falling within genic regions in the two MEF cells showed evidence of transcription-coupled repair, with a lower fraction of ENU-induced mutations occurring at T and G bases, the predominant adduct bases, on the transcribed strand than the non-transcribed strand (FIG. 4C). This bias appeared strongest for T>A transversions, supporting previous results at the endogenous HPRT gene locus (21). No evidence for any transcription-coupled repair process was seen in the S2 cells, in keeping with both experimental results (25,26) and the absence of homologues of either CSA or CSB (27), the main TCR genes, in the mouse.

In summary, these results show for the first time how massively parallel sequencing can be used effectively for measuring random, low-abundance mutations in somatic cells. Of note, while this work was entirely focused on DNA point mutations, also detected were other types of mutations, such as small indels. Also structural variation could be detected, using the paired-end sequencing approach in Drosophila S2 cells (not shown). To date genome-wide studies of mutagenesis have been concerned only with identifying mutations in clones, for example, by whole genome sequencing of tumors. The single cell approach taken in this study opens up the possibility to study low-abundance mutations within tissues, most notably pre-neoplastic or neoplastic tissues (28). Tumors are genomically heterogeneous with each cell carrying its own unique capabilities for growing into a full-blown tumor (29,30). The ability to analyze subclonal genetic diversity will greatly expand the possibility to obtain important clinical information about a particular cancer in a particular patient.

Finally, the methodology for the first time provides a direct approach for estimating individual risk of exposure to mutagenic agents, such as radiation. DNA mutation is the critical end point for cancer, the main long-term adverse health effect of environmental mutagens. Currently there are no methods to directly assess DNA mutation loads in exposed individuals. Genome-wide sequence analysis of a representative number of cells from a blood sample or tissue biopsy according to the procedures outlined in this work provides such a method.

### Methods and Materials

Single cells were collected under an inverted microscope by hand-held capillaries, deposited in PCR tubes along with 2 μl of culture medium, and immediately frozen on dry ice. Cells were lysed and amplified using the REPLI-g UltraFast Mini kit (Qiagen, Santa Clara, Calif.) according to the manufacturer's instructions, but using an initial 30-min amplification at 30° C. followed by an 18-hour amplification at 35° C. The reaction products were purified using AMPure XP magnetic beads (Agencourt, Beverly, Mass.) and the reaction yield was measured using the NanoDrop 1000 spectrophotometer (Nanodrop Technologies LLC, Wilmington, Del.). Reactions with yield of greater than 1 μg were tested for locus dropout at eight loci using comparative Ct measurements from real-time PCR (StepOne Plus, Applied Biosystems, Foster City, Calif.) performed with Fast SYBR® Green Master Mix (Foster City, Calif.). Up to 5 μg of DNA from samples displaying the least biased amplification was used as input for Illumina libraries. DNA was either randomly fragmented (S2 cells) or digested (MEFs) with 50 U of Msei (NEB, Ipswich, Mass.). Digested DNA (MEFs) was end-repaired using Mung Bean Nuclease (NEB, Ipswich, Mass.) and then used as input for the Illumina library preparation. S2 libraries were size-selected to 475-525-bp and MEF libraries were size selected to 250-350-bp using agarose gel electrophoresis. Libraries were sequenced using 108-bp paired-end sequencing (S2 cells) or 118-bp single-end sequencing (MEFs) on the HiSeq 2000 (Illumina, San Diego, Calif.). Raw sequencing data was aligned to the dm3 (S2 cells) and mm9 (MEFs) reference sequences using BWA with standard parameters. The aligned sequence data was processed using genome analysis toolkit (GATK) (e.g., available at www.broadinstitute.org) to realign reads containing indels or a high entropy of mismatches, recalibrate the base quality scores, and to compute coverage data and statistics. Somatic point mutations and germline variation were scored using a pipeline composed of SAMtools mpileup command (e.g., available at samtools.sourceforge.net/mpileup.shtml), VarScan somatic command (e.g., available at varscan.sourceforge.net/somatic-calling.html) and a custom script to parse and filter the VarScan output. Somatic events found in multiple single cells were discarded, as were events found in at least one read in the unamplified control sample. Filtered somatic point mutations were visually validated using a custom IGV batch script (IGV is available at, e.g., www.broadinstitute.org) that recorded images of aligned reads at each locus containing a somatic point mutation. Analysis of the localization and spectra of point mutations was performed using GATK.

### REFERENCES

1 Lynch, M. Evolution of the mutation rate. Trends Genet 26, 345-352, doi:110.1016/j.tig.2010.05.003 (2010).
2 Durbin, R. M. et al. A map of human genome variation from population-scale sequencing. Nature 467, 1061-1073, doi:10.1038/nature09534 (2010).
3 Mills, R. E. et al. Mapping copy number variation by population-scale genome sequencing. Nature 470, 59-, 595 doi:10.038/nature09708 (2011).
4 Comprehensive genomic characterization defines human glioblastoma genes and core pathways. Nature 455, 1061-1068, doi:10.1038/nature07385 (2008).
5 Harismendy, O. et al. Evaluation of next generation sequencing platforms for population targeted sequencing studies. Genome Biol 10, R32, doi:10.1186/gb-2009-10-3-r32 (2009).
6 Bielas, J. H. & Heddle, J. A. Proliferation is necessary for both repair and mutation in transgenic mouse cells. Proceedings of the National Academy of Sciences of the United States of America 97, 11391-11396, doi:10.1073/pnas.190330997 (2000).
7 Mientjes, E. J. et al. Formation and persistence of O6-ethylguanine in genomic and transgene DNA in liver and brain of lambda(lacZ) transgenic mice treated with N-ethyl-N-nitrosourea. Carcinogenesis 17, 2449-2 454 (1996).
8 Mahabir, A. G. et al. lacZ mouse embryonic fibroblasts detect both clastogens and mutagens. Mutation research 666, 50-56, doi:10.1016/j.rmrfmmm.2009.04.005 (2009).
9 Lasken, R. S. Genomic DNA amplification by the multiple displacement amplification (MDA) method. Biochem Soc Trans 37, 450-453, doi:10.1042/BST0370450 (2009).
10 Li, H. & Durbin, R. Fast and accurate short read alignment with Burrows-Wheeler transform. Bioinformatics 25, 1754-1760, doi:10.1093/bioinformatics/btp324 (2009).
11 Depristo, M. A. et al. A framework for variation discovery and genotyping using next-generation DNA sequencing data. Nat Genet, doi:0.01038/ng.806 (2011).
12 Robinson, J. T. el at. Integrative genomics viewer. Nat Biotechnol 29, 24-26, doi:10.1038/nbt.1754 (2011).
13 Zhang, Y. et al. Expression in aneuploid Drosophila S2 cells. PLoS Biol 8, e1000320, doi:10.1371/journal.pbio.1000320 (2010).
14 Busuttil, R. A., Rubio, M., Dolle, M. E., Carnpisi, J. & Vijg, J. Oxygen accelerates the accumulation of mutations during the senescence and immortalization of murine cells in culture. Aging Cell 2, 287-294 (2003).
15 Dusenbery, R. L. & Smith, P. D. Cellular responses to DNA damage in Drosophila melanogaster. Mutation research 364, 133-145 (1996).
16 Kondo, N., Takahashi, A., Ono, K. & Ohnishi, T. DNA damage induced by alkylating agents and repair pathways. J Nucleic Acids 2010, 543531, doi:10.4061/2010/543531 (2010).
17 Vogel, E. W. & Natarajan, A. T. DNA damage and repair in somatic and germ cells in vivo. Mutation research 330, 183-208 (1995).
18 Tosal, L., Comendador, M. A. & Sierra, L. M. In vivo repair of ENU-induced oxygen alkylation damage by the nucleotide excision repair mechanism in Drosophila melanogaster. Mol Genet Genomics 265, 327-335 (2001).
19 Jansen, J. G. et al. Molecular analysis of hprt gene mutations in skin fibroblasts of rats exposed in vivo to N-methyl-N-nitrosourea or N-ethyl-N-nitrosourea. Cancer Res 54, 2478-2485 (1994).
20 Op het Veld, C. W., van Hees-Stuivenberg, S., van Zeeland, A. A. & Jansen, J. G. Effect of nucleotide excision repair on hprt gene mutations in rodent cells exposed to DNA ethylating agents. Mutagenesis 12, 417-424 (1997).
21 Skopek, T. R., Walker, V. E., Cochrane, J. E., Craft, T. R. & Cariello, N. F. Mutational spectrum at the Hprt locus in splenic T cells of B6C3F1 mice exposed to N-ethyl-N-nitrosourea. Proceedings of the National Academy of Sciences of the United States of America 89, 7866-7870 (1992).
22 Walker, V. E. et al. Frequency and spectrum of ethylnitrosourea-induced mutation at the hprt and lacI loci in splenic lymphocytes of exposed lacI transgenic mice. Cancer Res 56, 4654-4661 (1996).
23 Bell, O. et al. Accessibility of the Drosophila genome discriminates PcG repression, H4K16 acetylation and replication timing. Nat Struct Mol Biol 17, 894-900, doi:10.1038/nsmb.1825 (2010).
24 Eaton, M. L. et al Chromatin signatures of the Drosophila replication program. Genome Res 21, 164-174, doi:10.1101/gr.116038.110 (2011).
25 Keightley, P. D. et al. Analysis of the genome sequences of three Drosophila melanogaster spontaneous mutation accumulation lines. Genome Res 19, 1195-1201, doi:10.1101/gr.091231.109 (2009).
26 de Cock, J. G. et al. Repair of UV-induced (6-4)photoproducts measured in individual genes in the Drosophila embryonic Kc cell line. Nucleic acids research 20, 4789-4793 (1992).
27 Sekelsky, J. J., Brodsky, M. H. & Burtis, K. C. DNA repair in Drosophila: insights from the Drosophila genome sequence. J Cell Biol 150, F31-36 (2000).
28 Navin, N. et al. Tumour evolution inferred by single-cell sequencing. Nature 472, 90-94, doi:10.1038/nature09807 (2011).
29 Salk, J. J., Fox, E. J. & Loeb, L. A. Mutational heterogeneity in human cancers: origin and consequences. Annu Rev Pathol 5, 51-75, doi:110.1146/annurev-pathol-121808-102113 (2010).
30 Salk, J. J. & Horwitz, M. S. Passenger mutations as a marker of clonal cell lineages in emerging neoplasia. Semin Cancer Biol 20, 294-303, doi:10.1016/j.semcancer.2010.10.008 (2010).

1. A method for determining if an agent increases somatic mutations in a genome of a cell, tissue, or subject exposed to the agent comprising:
a) amplifying a first sample of genomic nucleic acid obtained from a cell, tissue, or subject prior to the cell, tissue, or subject, respectively, being exposed to the agent;
b) either (i) randomly fragmenting the nucleic acid sample into fragments or (ii) generating a range of fragments of the nucleic acids from the sample amplified in step a) using one or more restriction enzymes,
and then sequencing the resultant fragments;
c) mapping the fragments sequenced in step b) to a reference nucleic acid sequence;
d) comparing the sequences of fragments mapped in step c) to a corresponding portion of the reference nucleic acid sequence so as to identify, and, optionally, quantify, mutation(s), indels and/or genome rearrangements in the genomic nucleic acid of the first sample;
e) amplifying a second sample of genomic nucleic acid obtained from the cell, tissue, or subject, respectively, after the cell, tissue, or subject, respectively, has been exposed to the agent;
f) either (i) randomly fragmenting the nucleic acid sample into fragments or (ii) generating a range of fragments of the nucleic acids from the sample amplified in step e) using one or more restriction enzymes,
and then sequencing the resultant fragments;
g) mapping the fragments sequenced in step f) to the reference nucleic acid sequence;
h) comparing the sequences of fragments mapped in step g) to a corresponding portion of the reference nucleic acid sequence so as to identify, and, optionally, quantify, mutation(s), indels and/or genome rearrangements in the genomic nucleic acid of the first sample;
i) comparing the number of mutations, indels and/or genome rearrangements identified or quantified in step h) with the number of mutations, indels and/or genome rearrangements identified or quantified in step d) for one or more sequences, or portion thereof, mapped in both step c) and step g),
wherein an increase in the number of mutations, indels and/or genome rearrangements identified or quantified in step h) compared to step d) indicates that the agent increases somatic mutations, indels and/or genome rearrangements in the genome of the cell, tissue, or subject, respectively, exposed to the agent, and wherein no change or a decrease in the number of mutations, indels and/or genome rearrangements identified or quantified in step h) compared to step d) indicates that the agent does not increase somatic mutations, indels and/or genome rearrangements in the genome of the cell, tissue, or subject, respectively, exposed to the agent.

2. (canceled)

3. The method of claim 1, wherein the amplifying is whole genome amplification.

4. The method of claim 1, wherein in step b) and/or in step f) the fragments are sequenced by paired-end sequencing.

5. (canceled)

6. The method of claim 1, further comprising in each of steps b) and f) size-selecting fragments before sequencing.

7. (canceled)

8. The method of claim 1, further comprising screening the amplified genome for locus dropout.

9. The method of claim 8, wherein screening the amplified genome for locus dropout is effected by using primer pairs distributed over different chromosomes and qPCR.

10. The method of claim 1, wherein the subject is a human subject.

11. The method of claim 10, wherein the subject has cancer and the agent is a chemotherapeutic.

12. The method of claim 1, comprising in step b) sequencing a locus on the fragments a plurality of times and selecting a consensus sequence of the resultant plurality of sequencing results as the fragment sequence mapped in step c) and compared to in step d).

13. The method of claim 1, comprising in step f) sequencing a locus on the fragments a plurality of times and selecting a consensus sequence of the resultant plurality of sequencing results as the fragment sequence mapped in step g) and compared to in step h).

14. A method for determining if an agent increases somatic mutations in a genome of a cell, tissue or subject exposed to the agent, comprising:
a) contacting a first sample of genomic nucleic acid, obtained from the cell, tissue, or subject before exposure to the agent, with a restriction enzyme under conditions permitting the restriction enzyme to cleave the genomic nucleic acid into a first plurality of fragments;
b) sequencing in whole, or in part, fragments produced in step a) of a predetermined length range;
c) mapping paired-end fragments sequenced in step b) to a reference nucleic acid sequence;
d) comparing the sequences of the paired-end fragments mapped in step c) to a corresponding portion of the reference nucleic acid sequence so as to identify mutation(s), indels and/or genome rearrangements in the genomic nucleic acid;
e) contacting a second sample of genomic nucleic acid, obtained from the cell, tissue or subject after the cell, tissue or subject, respectively, has been exposed to the agent, with a restriction enzyme under conditions permitting the restriction enzyme to cleave the genomic nucleic acid into a second plurality of fragments;
f) sequencing in whole, or in part, fragments produced in step e) which are of the predetermined length range;
g) mapping paired-end fragments sequenced in step f) to the reference nucleic acid sequence;
h) comparing the sequences of fragments mapped in step g) to a corresponding portion of the reference nucleic acid sequence so as to identify mutation(s), indels and/or genome rearrangements in the genomic nucleic acid of the second sample after exposure to the agent; and
i) comparing the number of mutations, indels and/or genome rearrangements identified in step h) with the number identified in step d) for one or more sequences, or portion thereof, mapped in both step c) and step g),
wherein an increase in the number of mutations, indels and/or genome rearrangements identified in step h) compared to step d) indicates that the agent increases somatic mutations, indels and/or genome rearrangements in the genome of the cell, tissue or subject, respectively, exposed to the agent, and wherein no change or a decrease in the number of mutations, indels and/or genome rearrangements identified in step h) compared to step d) indicates that the agent does not increase somatic mutations, indels and/or genome rearrangements in the genome of the cell, tissue, or subject, respectively, exposed to the agent.

15-18. (canceled)

19. The method of claim 1, wherein the time between the end of exposure of the cell, tissue or subject to the agent and the beginning of step e) is at least one hour, at least one day, at least one week, at least one month or at least one year.

20-22. (canceled)

23. The method of claim 1, wherein, in steps d) and h), the number of mutations is quantified.

24. The method of claim 1, further comprising discounting all rearrangement artifacts from the number of mutations quantified.

25-26. (canceled)

27. The method of claim 1, wherein the nucleic acid is a single cell genome.

28-29. (canceled)

30. The method of claim 1, wherein the reference nucleic acid sequence is a human genome set forth in hg19 or is a custom reference sequence determined from a predetermined cell, tissue or subject of the same type as the cell, tissue or subject the nucleic acid sample was obtained from.

31. The method of claim 1, further comprising, after mapping paired-end sequenced fragments, discarding sequences having a mapping quality score below a predetermined value prior to comparing the sequences of the remaining fragments to the corresponding portions of the reference nucleic acid sequence.

32. The method of claim 1, further comprising, after mapping paired-end sequenced fragments, discarding chimeric sequences, wherein a sequence is determined as chimeric through application of an algorithm that uses an in silico digestion to define a chimeric signature as occurring between two fragments selected for during restriction digestion and subsequent predetermined length selection.

33-46. (canceled)

47. A system for performing the method of claim 1, comprising:
one or more data processing apparatus; and
a computer-readable medium coupled to the one or more data processing apparatus having instructions stored thereon which, when executed by the one or more data processing apparatus, cause the one or more data processing apparatus to perform the method.

48-49. (canceled)

50. The method of claim 1, wherein the fragments represent up to 2% of the genome.

51-55. (canceled)


SEQUENCE LISTING (3 sequences)

SEQ ID NO: 1 - DNA, 22 bp, Artificial Sequence
  THEORETICAL SEQUENCE FOR ILLUSTRATIVE PURPOSES ONLY, NOT PURPOSELY DERIVED FROM ANY SPECIES
catttagtttgatgttggctat

SEQ ID NO: 2 - DNA, 40 bp, Artificial Sequence
  DERIVED FROM S2 INSECT CELL LINE
catcactggcatggccatcggcaccggcagcgatatggga

SEQ ID NO: 3 - DNA, 40 bp, Artificial Sequence
  SEQUENCE DERIVED FROM S2 INSECT CELL LINE
ttcccatatcgctgcccgtgccgatggccatgccagtgat